(19) United States
(12) Patent Application Publication
(10) Pub. No.: US 2023/0285500 A1
(43) Pub. Date: Sep. 14, 2023

(54) Compositions and Methods for Gene Therapy for Corneal Endothelium Disorders

(71) Applicant: The Schepens Eye Research Institute, Inc., Boston, MA (US)

(72) Inventor: Ula V. Jurkunas, Winchester, MA (US)

(21) Appl. No.: 18/172,062

(22) Filed: Feb. 21, 2023

Related U.S. Application Data
(63) Continuation of application No. 16/825,311, filed on Mar. 20, 2020.
Application No. 16/825,311 is a continuation of application No. 13/809,996, filed on Jul. 17, 2013, now abandoned.
(60) Provisional application No. 61/364,605, filed on Jul. 15, 2010.
(60) Provisional application No. 61/482,769, filed on May 5, 2011.

(51) Int. Cl. A61K 38/07; A61K 9/00; A61K 31/26; A61K 45/06; A61K 31/00; A61K 31/385

(52) U.S. Cl. CPC A61K 38/07; A61K 9/0048; A61K 31/26; A61K 45/06; A61K 31/00; A61K 31/385

(57) ABSTRACT

This application discloses compositions and methods for treating corneal endothelium disorders (e.g., Fuchs endothelial corneal dystrophy) with one or more Nrf2 activators and/or mitochondrially targeted antioxidants using gene therapy.

### RELATED APPLICATIONS

This application is a continuation of U.S. patent application Ser. No. 16/825,311, filed Mar. 20, 2020, which is a continuation of U.S. patent application Ser. No. 13/809,996 filed Jul. 17, 2013, now abandoned, which is a U.S. national stage application, filed under 35 U.S.C. § 371, of International Application No. PCT/US2011/042664, filed Jun. 30, 2011, which claims the benefit of U.S. Provisional Application No. 61/482,769, filed May 5, 2011, and U.S. Provisional Application No. 61/364,605, filed Jul. 15, 2010. The contents of each of these applications are hereby incorporated by reference in their entireties.

### STATEMENT REGARDING FEDERALLY SPONSORED RESEARCH

This invention was made with Government support under Grant Number EY016335 awarded by the National Institutes of Health (NIH). The government has certain rights in the invention.

### FIELD OF THE DISCLOSURE

The present invention relates to compositions and methods for the treatment of corneal endothelium disorders.

### BACKGROUND OF THE DISCLOSURE

Fuchs endothelial corneal dystrophy (FECD) is a progressive, blinding disease characterized by corneal endothelial (CE) cell apoptosis. Corneal transplantation is the only measure currently available to restore vision. Despite the identification of some genetic factors, the pathophysiology of FECD remains unclear. There exists a need for topical ophthalmic pharmaceutical products to effectively treat FECD.

FECD causes gradual death of corneal endothelial cells and concomitant formation of extracellular excrescences, called guttae. Loss of endothelial cells leads to corneal edema and loss of vision. FECD affects approximately 1% of general population, while corneal guttae can be detected in about 4-6% of general population. Even though this dystrophy has been described in early 1900's, there is no known treatment for this disorder, and the only modality that restores lost vision is corneal transplantation. FECD is the second most common cause for corneal transplants done in the U.S. in >50 year-old age group. Development of pharmacotherapeutics that could prevent endothelial cell loss in early, as well as late stages of the disease would address one of most pressing unmet medical needs in ophthalmology.

### SUMMARY OF THE INVENTION

The invention relates to pharmaceutical and topical pharmaceutical compositions (e.g., oral or topical ophthalmic formulations) to treat diseased or damaged corneal endothelium of a subject in need of such treatment. For example, the methods involve systematically administering a pharmaceutical composition (e.g., orally) or by locally administering a pharmaceutical composition (e.g., topical ophthalmic formulations) directly to the eye of the subject. Diseases and conditions of the corneal endothelium include, but are not limited to, pseudophakic bullous keratopathy (PBK), Fuchs endothelial corneal Dystrophy (FECD), posterior polymorphous dystrophy, iridocorneal endothelial (ICE) syndrome, and congenital hereditary endothelial dystrophy (CHED).

Pharmaceutical compositions/formulations comprise at least one Nrf2 activator or agonist, alone or in combination with one or more additional active agents. For example, the Nrf2 agonist comprises sulforaphane (SF) and/or 3H-1,2-dithiole-3-thione (D3T), each of which is administered at approximately 50 micromolar for cells, e.g., about 5 micromolar, about 10 micromolar, about 25 micromolar, about 75 micromolar, or about 100 micromolar. N-acetylcysteine and mitochondrial antioxidants are also suitable Nrf2 activators/agonists. Exemplary antioxidants that are used alone or in combination with Nrf2 agonists include Vitamin C, Vitamin E, Carotenoids, and Retinols. Other Nrf2 agonists, such as statins, are also suitable to upregulate Nrf2. The combination formulations of Nrf2 activators are effective in treating the corneal endothelium. In some embodiments, the at least one Nrf2 activator and/or other active ingredients may be added to irrigation solutions, such as those irrigation solutions routinely used during phacoemulcification, vitreoretinal, and/or intraocular procedures.

In some embodiments, there are provided pharmaceutical compositions (e.g., oral or topical ophthalmic formulations) of at least one Nrf2 activator, alone or in combination with one or more additional active agents. The combination formulations of Nrf2 activators are effective in treating the corneal endothelium.

In some embodiments, methods are provided for treating the corneal endothelium by administering one or more Nrf2 activators to the eye of a subject in need of such treatment or prevention. In some embodiments, kits are provided comprising a pharmaceutical composition of one or more Nrf2 activator formulated for ophthalmic use and instructions for such use.

In some embodiments, the invention provides a method for treating the corneal endothelium by administering to a subject in need thereof therapeutically effective amount of one or more Nrf2 activators or Nrf2 agonists, e.g., statins. The one or more Nrf2 activators may be administered using any route of administration (e.g., orally and parenterally). The Nrf2 activators described herein are also combined with corneal storage media such as Optisol to enhance endothelial cell survival prior to and during transplantation.

In one embodiment, the invention provides a method for treating the corneal endothelium by administering to the eye of a subject in need thereof an ophthalmic formulation comprising an effective amount of one or more Nrf2 activators. In certain embodiments, the concentration of Nrf2 activators may be from 0.10% to 5.0% (w/v), preferably from 0.10% to 2.0%.

According to some embodiments, Nrf2 activators are selected from the groups consisting of hydroquinones, sulforaphanes, polyphenols (such as reservatol), curcumins, and catecols and combinations and mixtures thereof. Thus, in some embodiments, methods and pharmaceutical compositions (e.g., oral or topical ophthalmic formulations) are provided for treating the corneal endothelium, wherein the formulation further comprises at least one hydroquinone. In some embodiments, methods and pharmaceutical compositions (e.g., oral or topical ophthalmic formulations) are provided for treating the corneal endothelium, wherein the formulation further comprises at least one sulforaphane. In some embodiments, methods and pharmaceutical compositions (e.g., oral or topical ophthalmic formulations) are provided for treating the corneal endothelium, wherein the formulation further comprises at least one polyphenol. In some embodiments, methods and pharmaceutical compositions (e.g., oral or topical ophthalmic formulations) are provided for treating the corneal endothelium, wherein the formulation further comprises at least one curcumin. In some embodiments, methods and pharmaceutical compositions (e.g., oral or topical ophthalmic formulations) are provided for treating the corneal endothelium, wherein the formulation further comprises at least one catecol.

According to some embodiments, there is provided a method for treating the corneal endothelium by administering to the eye of a subject in need thereof an ophthalmic formulation comprising an effective amount of one or more Nrf2 activators, a pharmacologically active derivative or analog thereof. Optionally, the compounds described herein are added into existing or new vitamin formulations to arrest the progression of endothelial aging and cell loss. The concentration of Nrf2 activators may be from 0.10% to 5.0% (w/v). The ophthalmic formulation may further comprise one or more mitochondrially targeted antioxidants. The ophthalmic formulation may further comprise one or more tear substitutes. In some embodiments, the at least one of the tear substitutes contains an ophthalmic lubricant (e.g., hydroxypropylmethylcellulose).

According to some embodiments, there is provided a method for treating the corneal endothelium by administering to the eye of a subject in need thereof an ophthalmic formulation comprising an effective amount of one or more mitochondrially targeted antioxidants, a pharmacologically active derivative or analog thereof. The concentration of mitochondrially targeted antioxidants may be from 0.10% to 5.0% (w/v). The ophthalmic formulation may further comprise one or more Nrf2 activators. The ophthalmic formulation may further comprise one or more tear substitutes. In some embodiments, the at least one of the tear substitutes contains an ophthalmic lubricant (e.g., hydroxypropylmethylcellulose).

In some embodiments, the one or more mitochondrially targeted antioxidants and/or other active ingredients may be added to irrigation solutions, such as those irrigation solutions routinely used during phacoemulcification, vitreoretinal, and/or intraocular procedures.

In a preferred embodiment of the method for treating the corneal endothelium, the subject is human.

The present invention relates to pharmaceutical compositions (e.g., oral or topical pharmaceutical compositions (e.g., oral or topical ophthalmic formulations)) useful to treat or mitigate the symptoms of FECD. Thus, the invention also provides methods for the treatment of FECD in a subject in need of such treatment by administering the pharmaceutical compositions (e.g., oral or topical ophthalmic formulations) of the present invention directly to the eye of the subject.

In some embodiments, there is provided pharmaceutical compositions (e.g., oral or topical ophthalmic formulations) comprising at least one Nrf2 activator, alone or in combination with one or more additional active agents. The combination formulations of Nrf2 activators are effective in treating or mitigating the symptoms of FECD.

In some embodiments, methods are provided for treating and preventing the symptoms of FECD by administering one or more Nrf2 activators to the eye of a subject in need of such treatment or prevention. In some embodiments, kits are provided comprising a pharmaceutical composition of one or more Nrf2 activator formulated for ophthalmic use and instructions for such use.

In one embodiment, the invention provides a method for treating FECD by administering to the eye of a subject in need thereof an ophthalmic formulation comprising an effective amount of one or more Nrf2 activators. In certain embodiments, the concentration of Nrf2 activators may be from 0.10% to 5.0% (w/v), preferably from 0.10% to 2.0%.

According to some embodiments, Nrf2 activators are selected from the groups consisting of hydroquinones, sulforaphanes, polyphenols (such as reservatol), curcumins, and catecols and combinations and mixtures thereof. Thus, in some embodiments, methods and pharmaceutical compositions (e.g., oral or topical ophthalmic formulations) are provided for treating FECD wherein the compositions further comprises at least one hydroquinone. In some embodiments, methods and pharmaceutical compositions (e.g., oral or topical ophthalmic formulations) are provided for treating FECD wherein the compositions further comprises at least one sulforaphane. In some embodiments, methods and pharmaceutical compositions (e.g., oral or topical ophthalmic formulations) are provided for treating FECD wherein the compositions further comprises at least one polyphenol. In some embodiments, methods and pharmaceutical compositions (e.g., oral or topical ophthalmic formulations) are provided for treating FECD wherein the compositions further comprises at least one curcumin. In some embodiments, methods and pharmaceutical compositions (e.g., oral or topical ophthalmic formulations) are provided for treating FECD wherein the compositions further comprises at least one catecol.

According to some embodiments, there is provided a method for treating FECD by administering to the eye of a subject in need thereof an ophthalmic formulation comprising an effective amount of one or more Nrf2 activators, a pharmacologically active derivative or analog thereof. The concentration of Nrf2 activators may be from 0.10% to 5.0% (w/v). The ophthalmic formulation may further comprise one or more mitochondrially targeted antioxidants. The ophthalmic formulation may further comprise one or more tear substitutes. In some embodiments, the at least one of the tear substitutes contains an ophthalmic lubricant (e.g., hydroxypropylmethylcellulose).

According to some embodiments, there is provided a method for treating FECD by administering to the eye of a subject in need thereof an ophthalmic formulation comprising an effective amount of one or more mitochondrially targeted antioxidants, a pharmacologically active derivative or analog thereof. The concentration of mitochondrially targeted antioxidants may be from 0.10% to 5.0% (w/v). The ophthalmic formulation may further comprise one or more Nrf2 activators. The ophthalmic formulation may further comprise one or more tear substitutes. In some embodiments, the at least one of the tear substitutes contains an ophthalmic lubricant (e.g., hydroxypropylmethylcellulose).

In a preferred embodiment of the method for treating FECD, the subject is human.

The present invention relates to topical pharmaceutical compositions (e.g., oral or topical ophthalmic formulations) for useful to treat or mitigate the symptoms of PBK. Thus, the invention also provides methods for the treatment of PBK in a subject in need of such treatment by administering the pharmaceutical compositions (e.g., oral or topical ophthalmic formulations) of the present invention directly to the eye of the subject.

In some embodiments, there is provided pharmaceutical compositions (e.g., oral or topical ophthalmic formulations) of at least one Nrf2 activator, alone or in combination with one or more additional active agents. The combination formulations of Nrf2 activators are effective in treating or mitigating the symptoms of PBK.

In some embodiments, methods are provided for treating and preventing the symptoms of PBK by administering one or more Nrf2 activators to the eye of a subject in need of such treatment or prevention. In some embodiments, kits are provided comprising a pharmaceutical composition of one or more Nrf2 activator formulated for ophthalmic use and instructions for such use.

In one embodiment, the invention provides a method for treating PBK by administering to the eye of a subject in need thereof an ophthalmic formulation comprising an effective amount of one or more Nrf2 activators. In certain embodiments, the concentration of Nrf2 activators may be from 0.10% to 5.0% (w/v), preferably from 0.10% to 2.0%.

According to some embodiments, Nrf2 activators are selected from the groups consisting of hydroquinones, sulforaphanes, polyphenols (such as reservatol), curcumins, and catecols and combinations and mixtures thereof. Thus, in some embodiments, methods and pharmaceutical compositions (e.g., oral or topical ophthalmic formulations) are provided for treating PBK wherein the formulation further comprises at least one hydroquinone. In some embodiments, methods and pharmaceutical compositions (e.g., oral or topical ophthalmic formulations) are provided for treating PBK wherein the formulation further comprises at least one sulforaphane. In some embodiments, methods and pharmaceutical compositions (e.g., oral or topical ophthalmic formulations) are provided for treating PBK wherein the formulation further comprises at least one polyphenol. In some embodiments, methods and pharmaceutical compositions (e.g., oral or topical ophthalmic formulations) are provided for treating PBK wherein the formulation further comprises at least one curcumin. In some embodiments, methods and pharmaceutical compositions (e.g., oral or topical ophthalmic formulations) are provided for treating PBK wherein the formulation further comprises at least one catecol.

According to some embodiments, there is provided a method for treating PBK by administering a subject in need thereof therapeutically effective amount of one or more Nrf2 activators, a pharmacologically active derivative or analog thereof. In some embodiments, the method further comprises administering one or more mitochondrially targeted antioxidants.

According to some embodiments, there is provided a method for treating PBK by administering to the eye of a subject in need thereof a therapeutically effective amount of one or more mitochondrially targeted antioxidants, a pharmacologically active derivative or analog thereof. In some embodiments, the method further comprises administering one or more Nrf2 activators.

According to some embodiments, there is provided a method for treating PBK by administering to the eye of a subject in need thereof an ophthalmic formulation comprising an effective amount of one or more Nrf2 activators, a pharmacologically active derivative or analog thereof. The concentration of Nrf2 activators may be from 0.10% to 5.0% (w/v). The ophthalmic formulation may further comprise one or more mitochondrially targeted antioxidants. The ophthalmic formulation may further comprise one or more tear substitutes. In some embodiments, the at least one of the tear substitutes contains an ophthalmic lubricant (e.g., hydroxypropylmethylcellulose).

According to some embodiments, there is provided a method for treating PBK by administering to the eye of a subject in need thereof an ophthalmic formulation comprising an effective amount of one or more mitochondrially targeted antioxidants, a pharmacologically active derivative or analog thereof. The concentration of mitochondrially targeted antioxidants may be from 0.10% to 5.0% (w/v). The ophthalmic formulation may further comprise one or more Nrf2 activators. The ophthalmic formulation may further comprise one or more tear substitutes. In some embodiments, the at least one of the tear substitutes contains an ophthalmic lubricant (e.g., hydroxypropylmethylcellulose).

In a preferred embodiment of the method for treating PBK, the subject is human.

In one embodiment, the pH of the formulation is between 5.5 and 7. In one embodiment, the formulation is an aqueous formulation. In one embodiment, the formulation is in the form of a single dose unit. In a specific embodiment, the single dose unit does not comprise a preservative. In certain embodiments, the formulation further comprises one or more tear substitutes. Preferably, at least one of the tear substitutes contains and ophthalmic lubricant (e.g., hydroxypropylmethylcellulose).

According to some embodiments, there is provided ophthalmic formulations comprising one or more Nrf2 activators, wherein the formulation is suitable for administration to the eye of a subject. The ophthalmic formulation may have a pH between 5.5 and 7. In some embodiments the ophthalmic formulation is an aqueous formulation. In some embodiments the ophthalmic formulation is in the form of a single dose unit. In some embodiments the ophthalmic formulation is does not comprise a preservative. The ophthalmic formulation may further comprise one or more mitochondrially targeted antioxidants. The ophthalmic formulation may further comprise one or more tear substitutes. In some embodiments, the at least one of the tear substitutes contains an ophthalmic lubricant (e.g., hydroxypropylmethylcellulose).

According to some embodiments, there is provided ophthalmic formulations comprising one or more mitochondrially targeted antioxidants, wherein the formulation is suitable for administration to the eye of a subject. The ophthalmic formulation may have a pH between 5.5 and 7. In some embodiments the ophthalmic formulation is an aqueous formulation. In some embodiments the ophthalmic formulation is in the form of a single dose unit. In some embodiments the ophthalmic formulation is does not comprise a preservative. The ophthalmic formulation may further comprise one or more tear substitutes. In some embodiments, the at least one of the tear substitutes contains an ophthalmic lubricant (e.g., hydroxypropylmethylcellulose). The ophthalmic formulations may further comprise one or more Nrf2 activators.

According to some embodiments, there is provided ophthalmic formulations comprising one more mitochondrially targeted antioxidants and Nrf2 activators, wherein the formulation is suitable for administration to the eye of a subject. The ophthalmic formulation may have a pH between 5.5 and 7. In some embodiments the ophthalmic formulation is an aqueous formulation. In some embodiments the ophthalmic formulation is in the form of a single dose unit. In some embodiments the ophthalmic formulation is does not comprise a preservative. The ophthalmic formulation may further comprise one or more tear substitutes. In some embodiments, the at least one of the tear substitutes contains an ophthalmic lubricant (e.g., hydroxypropylmethylcellulose).

In some embodiments, at least one Nrf2 activator and/or mitochondrial protective agent may be administered using viral-based systems (e.g., a live viral expression system) that induce the genes of interest (e.g., Nrf2 (Genbank Accession No. NM 003204.2 (GI:189181670), incorporated herein by reference), DJ-1 (PARK7; Genbank Accession No. NM_007262.4 (GI:183227676), incorporated herein by reference), and Keap1 (Genbank Accession No. NM 203500.1 (GI:45269144), incorporated herein by reference)). Using this approach, recombinant DNA constructs are directly administered or targeted to the required site in vivo using appropriate viral vectors. A number of viral based systems have been developed for direct gene transfer into mammalian cells. In this regard, retroviruses provide a convenient platform for gene delivery systems. A selected nucleotide sequence encoding the desired polypeptide can be inserted into a vector and packaged in retroviral particles using techniques known in the art. The recombinant virus can then be isolated and delivered to a subject. A number of suitable retroviral systems have been described (U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109. Other features and advantages of the invention will become apparent from the following detailed description and claims.

Suitable virus-based expression systems for use in the present embodiments include: parvovirus; lentivirus (LV); retrovirus; adenovirus; herpesvirus; adeno-associate virus (AAV), including rAAV, AAV2 and AAV5; viral-mediated RNAi (siRNA); AdV-mediated expression of shRNA; LV-based delivery of shRNA; and viral-based reversible and irreversible regulated delivery of shRNA. See e.g., Raoul et al., Gene Therapy (2006) 13, 487-495 and Mah et al., Lin Pharmakokinet (2002) 41(12): 901-911, the entire contents of which are incorporated by reference herein in their entireties.

Ocular Irrigating Solutions

Nrf2 agonists can be added as an active ingredient to any ocular irrigation solution. Described herein are ocular irrigating solutions comprising an effective amount of an Nrf2 agonist or activator, a pharmacologically active derivative, or analog thereof. Optionally, the ocular irrigating solutions further include mitochondrially targeted antioxidants. The inclusion of Nrf2 agonists and/or mitochondrially targeted antioxidants enhance the effect of such ocular irrigation solutions, and prevent endothelial cell loss during surgery.

Also provided herein are methods of irrigating an eye or adnexal tissue of a subject comprising administering to the subject in need thereof an ocular irrigating solution comprising an effective amount of a Nrf2 agonist or activator, a pharmacologically active derivative, or analog thereof. Optionally, the ocular irrigating solution further includes a mitochondrially targeted antioxidant.

Optionally, the ocular irrigating solution is administered prior to a medical or surgical procedure to said eye or adnexal tissue, e.g., at least 24 hours, at least 12 hours, at least 6 hours, at least 2 hours, at least 1 hour, or at least 30 minutes prior to the medical or surgical procedure to the eye or adnexal tissue. The ocular irrigating solution is suitable for administration during a medical or surgical procedure to the eye or adnexal tissue. In another aspect, the ocular irrigating solution is administered after a medical or surgical procedure to the eye or adnexal tissue, e.g., at least 30 minutes, at least 1 hour, at least 2 hours, at least 6 hours, at least 12 hours, or at least 24 hours after a medical or surgical procedure to the eye or adnexal tissue. Alternatively, the ocular irrigating solution is administered in the absence of a medical or surgical procedure to the eye or adnexal tissue. Preferably, the ocular irrigating solution relieves irritation, stinging, discomfort, and/or itching, and removes loose foreign material, air pollutants, or chlorinated water.

The osmolality of the ocular irrigating solution is within 10% of that of human eye tissue, e.g., within 9%, within 8%, within 7%, within 6%, within 5%, within 4%, within 3%, within 2%, or within 1% of that of human eye tissue, e.g., aqueous humor. The pH of the ocular irrigating solution is within 0.18 pH units of, e.g., human aqueous humor. For example, the pH of the ocular irrigating solution is within 0.17, 0.16, 0.15, 0.14, 0.13, 0.12, 0.11, 0.10, 0.09, 0.08, 0.07, 0.06, 0.05, 0.04, 0.03, 0.02, or 0.01 pH units of human aqueous humor. The concentration of glutathione (mmol/L or mEq/L) in the ocular irrigating solution is within 0.5 mmol/L or mEq/L, e.g., within 0.4, 0.3, 0.25, 0.2, 0.15, 0.1, 0.5, or 0.1 mmol/L or mEq/L of that of human eye tissue, e.g., aqueous humor. The concentration of glucose (mmol/L or mEq/L) in the ocular irrigating solution is within 2 mmol/L or mEq/L of that of human eye tissue, e.g., within 1.9, 1.8, 1.5, 1.25, 1.0, 0.75, 0.5, 0.25, 0.1, 0.5, or 0.1 of that of human eye tissue, e.g., aqueous humor. The concentration of phosphate (mmol/L or mEq/L) in the ocular irrigating solution is within 2.5 mmol/L or mEq/L of human eye tissue, e.g., within 2.4, 2.3, 2.2, 2.0, 1.8, 1.6, 1.4, 1.2, 1.0, 0.08, 0.06, 0.04, or 0.02 mmol/L or mEq/L of that of human eye tissue, e.g., aqueous humor. The concentration of bicarbonate (mmol/L or mEq/L) in the ocular irrigating solution is within 5 mmol/L or mEq/L of human eye tissue, e.g., within 4.5, 4.0, 3.5, 3.0, 2.5, 2.0, 1.5, 1.0, or 0.5 of that of human eye tissue, e.g., aqueous humor. The concentration of chloride in the ocular irrigating solution is within 2 mmol/L or mEq/L of human eye tissue, e.g., within 1.8, 1.6, 1.4, 1.2, 1.0, 0.8, 0.6, 0.4, 0.2, or 0.1 mmol/L or mEq/L of that of human eye tissue, e.g., aqueous humor. The concentration of magnesium in the ocular irrigating solution is within 0.2 mmol/L or mEq/L of human eye tissue, e.g., within 0.15, 0.1, 0.5, or 0.1 mmol/L or mEq/L of that of human eye tissue, e.g., aqueous humor. The concentration of calcium in the ocular irrigating solution is within 1.2 mmol/L or mEq/L of human eye tissue, e.g., within 1.1, 1.0, 0.8, 0.6, 0.4, 0.2, or 0.1 mmol/L or mEq/L of that of human eye tissue, e.g., aqueous humor. The concentration of potassium in the ocular irrigating solution is within 1.2 mmol/L or mEq/L of human eye tissue, e.g., within 1.1, 1.0, 0.8, 0.6, 0.4, 0.2, or 0.1 mmol/L or mEq/L of that of human eye tissue, e.g., aqueous humor. Finally, the concentration of sodium in the ocular irrigating solution is within 160 mmol/L or mEq/L of human eye tissue, e.g., within 150, 125, 100, 75, 50, 25, 10, 5, 4, 3, 2, or 1 mmol/L or mEq/L of that of human eye tissue, e.g., aqueous humor.

As described herein, an ophthalmic irrigating solution is an extraocular and intraocular irrigating solution used before, during, or after ocular surgical procedures involving perfusion of the eye. Specifically, the lavage or eye rinse is performed prior to, during, or after a medical or surgical procedure to the eye or adnexal tissue of a subject. Alternatively, the ocular irrigation solution is used to flush irritants (e.g., foreign material, chlorinated water, or air pollutants such as smog, pollen, ragweed, or dust) from the eye. The ocular irrigation solutions of the invention are also used for extraocular irrigation following minor surgery or diagnostic procedures and for all cases where a non-irritating extraocular irrigating solution is needed. It is useful as a first aid emergency treatment for flushing chemicals (such as chlorine, weed killer, bleach or oven cleaner) from the eye(s) or following acid/alkaline burn of the eye. The ocular irrigation solution can also be used to soothe eye(s) that have been irritated by exposure to heat, smoke, fires or chemical fumes.

The affected eye(s) are flushed as needed with the ocular irrigation solution, controlling flow rate by varying pressure on the bottle. The ocular irrigation solutions described herein are gentle and suitable for use as often as needed to gently sooth eyes by relieving irritation, stinging, discomfort, and/or itching.

The ocular irrigation solutions of the invention are sterile, buffered, and isotonic. One example of an ocular irrigation solution is BSS® Sterile Irrigating Solution or BSS PLUS® by Alcon®. For example BSS PLUS® by Alcon® includes the components glutathione and ascorbate, which distinguish it from other sterile, buffered, isotonic, saline solutions. The inclusion of Nrf2 agonists and/or mitochondrially targeted antioxidants enhance the effect of such ocular irrigation solutions, and prevent endothelial cell loss during surgery.

The ingredients of BSS PLUS® and BSS® by Alcon® are set forth in the table below. All concentrations expressed in mmol/L or mEq/L solution.

| | | | | BSS | |
|---|---|---|---|---|---|
| | | | Hartman's | PLUS ^{®} | BSS ^{®} |
| | Human | Human | Lactated | Intraocular | Intraocular |
| | Aqueous | Vitreous | Ringer's | Irrigating | Irrigating |
| Ingredient | Humor | Humor | Solution | Solution | Solution |
| Sodium | 162.9 | 144 | 102 | 160.0 | 155.7 |
| Potassium | 2.2-3.9 | 5.5 | 4 | 5.0 | 10.1 |
| Calcium | 1.8 | 1.6 | 3 | 1.0 | 3.3 |
| Magnesium | 1.1 | 1.3 | — | 1.0 | 1.5 |
| Chloride | 131.6 | 177.0 | — | 130.0 | 128.9 |
| Bicarbonate | 20.15 | 15.0 | — | 25.0 | — |
| Phosphate | 0.62 | 0.4 | — | 3.0 | — |
| Lactate | 2.5 | 7.8 | 28 | — | — |
| Glucose | 2.7-3.7 | 3.4 | — | 5.0 | — |
| Ascorbate | 1.06 | 2.0 | — | — | — |
| Glutathione | 0.0019 | — | — | 0.3 | — |
| Citrate | — | — | — | — | 5.8 |
| Acetate | — | — | — | — | 28.6 |
| pH | 7.38 | — | 6.0-7.2 | 7.4 | 7.6 |
| Osmolality | 304 | — | 277 | 305 | 298 |
| (mOsm) | | | | | |

A suitable ocular solution comprises an Nrf2 agonist, purified water, and optionally other compounds such as boric acid, sodium borate, and sodium chloride. Optionally, the ocular irrigating solution further includes mitochondrially targeted antioxidants. Hydrochloric acid and/or sodium hydroxide is used to adjust pH to physiologic range. Suitable preservatives include edetate disodium, sorbic acid, and benzalkonium chloride, e.g., edetate disodium 0.025%, sorbic acid 0.1%, and benzalkonium chloride 0.013%. Exemplary buffering agents include sodium acetate trihydrate 0.39% and sodium citrate dihydrate 0.17%. Suitable tonicity agents (per mL) include sodium chloride 0.64%, potassium chloride 0.075%, calcium chloride dihydrate 0.048%, and magnesium chloride hexahydrate 0.03%.

Another example of an ocular irrigation solution comprises an Nrf2 agonist, sodium chloride (NaCl) 0.64%, potassium chloride (KCl) 0.075%, calcium chloride dihydrate (CaCl₂·H₂O) 0.048%, magnesium chloride hexahydrate (MgCl₂·6H₂O) 0.03%, sodium acetate trihydrate (C₂H₃NaO₂·3H₂O) 0.39%, sodium citrate dihydrate (C₆H₅Na₃O₇·2H₂O) 0.17%, sodium hydroxide and/or hydrochloric acid (to adjust pH), and water for injection. Optionally, the ocular irrigating solution further includes mitochondrially targeted antioxidants. The pH of the ocular irrigation solutions is approximately 7.0, while the osmolality is approximately 300 mOsm/Kg.

The active ingredients of the ocular irrigation solutions include an Nrf2 agonist or activator, a pharmacologically active derivative, or analog thereof, a mitochondrially targeted antioxidant, and purified water. Optionally, inactive ingredients in ocular irrigation solutions include boric acid, sodium borate and sodium chloride.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIGS. 1A-B. Relative expression of antioxidant and oxidative stress-related genes detected by PCR array in FECD and normal corneal endothelium. (A) Scatter plot shows the distribution of the mRNA fold-changes between five normal and five FECD samples relative to the housekeeping genes B2M, RPL13a and β-actin. Bold lines represent 2-fold change set as a threshold of up- and downregulation. Middle line represents fold-change of 1. (B) The data obtained from the PCR array is summarized in a scheme of the enzymatic antioxidant systems that reduce superoxide radical (O2-) and H₂O₂ to water. There are three forms of SOD, the main enzymes responsible for scavenging superoxide radical. PRDX5, catalase, and glutathione peroxidases are the primary enzymes responsible for scavenging H₂O₂. TXNRD1 catalyzes the regeneration of peroxiredoxins. Glutathione reductase, glutathione transferase, and glutathione synthetase are the components of the glutathione peroxidase system. The other antioxidant systems that scavenge H₂O₂ are MT3, cytoglobin (CYGB), and ALB. The arrows indicate which genes are overexpressed or underexpressed in FECD CE as compared to normal CE. Boldfaced genes had statistically significant fold changes in FECD CE as compared to normal.

FIGS. 2A-B. Decreased Nrf2 protein levels in FECD as compared to normal endothelium. (A) Western blot analysis of Nrf2 production in FECD and normal (N1) corneal endothelial samples. Mouse kidney extract was used as a positive control (C). Bands were detected at the appropriate molecular weight of 57 kDa. β-actin was used for normalization of protein loading. (B) Densitometric analysis of Nrf2 expression in CE. Data are means±SEM of four FECD and normal samples. Nrf2 protein levels were significantly decreased) in FECD CE as compared to normal controls, *P=0.02.

FIGS. 3A-B. Increased oxidative DNA damage in FECD as compared to normal CE. (A) High sensitivity ELISA was used to detect average concentration of 8-OHdG, an oxidative DNA damage marker, per nanogram of DNA from FECD patients and normal subjects. Data are means±SEM of five independent FECD and normal samples. The level of 8-OHdG in FECD CE was statistically significantly higher than in normal CE, *P=0.006. (B) Whole mount corneas with endothelium side up were labeled with MitoTracker, a mitochondrion-specific stain and 8-OHdG. Representative confocal images of normal (top row) and FECD CE (bottom row) in whole mounts of corneal tissue with endothelium side up. Images were taken after staining of mitochondria with MitoTracker Red (red) (first column) and immunolocalization of 8-OHdG (green) (second column). Images of negative controls incubated with only secondary antibody are shown in the right column. TOPRO-3 was used for nuclei staining (blue). Overlay of the three channels shows colocalization of MitoTracker and 8-OHdG (third column) in FECD. Asterisks (*) indicate the characteristic guttae of FECD CE. Original magnification 400× with 8× zoom.

FIGS. 4A-B. Effect of H₂O₂ treatment on the level of Nrf2 mRNA expression and oxidative DNA damage in HCECi. Corneal endothelial cells were treated with H₂O₂ (200 μM) for 2 hours in three independent experiments. (A) Real-time PCR analysis showed a decrease in Nrf2 expression after treatment with H₂O₂ (+H₂O₂) as compared to non-treated HCECi (control). Results were expressed as fold-changes and normalized to B2M mRNA expression. (B) High sensitivity ELISA detected an increase in the average level of oxidized DNA (8-OHdG) in +H₂O₂-treated cells as compared to control. Data are means±SEM, *P<0.05.

FIGS. 5A-C. Colocalization of apoptosis and oxidative DNA damage in FECD as compared to normal and PBK specimens. (A) Corneal endothelium attached to its native basement membrane from normal (top), FECD (middle), and PBK (bottom) donors was labeled with TUNEL (red), anti-8-OHdG (green), and TOPRO-3 (blue). Colocalization of TUNEL and anti-8-OHdG antibodies is detected in CE cells from FECD specimens but not from PBK and normal corneas. Asterisks (*) indicate the characteristic guttae of FECD CE. Magnification: 400× with 8×zoom. (B) Corneal endothelial cell density is significantly lower in FECD and PBK specimens. The percent of TUNEL-positive cells is higher in FECD. (C) Densitometric analysis of CE labeled with anti-8-OHdG antibody indicates a significant increase in oxidative damage in FECD but not PBK, as compared to normal controls. Data are means±SEM of four normal, FECD, and PBK samples, *P<0.05 compared with normal CE.

FIGS. 6A-C. (A) In vivo confocal microscopy photographs of corneal endothelium from normal and FECD patients. Normal endothelium exhibits regularly shaped hexagonal CE cells. In FECD, the CE cell mosaic is interrupted by guttae (arrowheads) and exhibits variable size (polymegethism) and variable shape (pleomorphism). (B) Mice corneal buttons were treated with H₂O₂-DMEM (0-100 μM) for 30 minutes. Confocal images of the whole mount corneas with CE cell junctions detected by ZO-1 (white) localization. (C) Automated cell analysis did not detect a change in CE cell density with increasing H₂O₂ concentrations, but the level of polymegethism (measured by coefficient of variation) and pleomorphism (measured by the number of hexagonal cells) was significantly altered after treatment with H₂O₂ at 50 μM or greater concentrations. Data are means±SD and are representative of four independent experiments; *P<0.05, compared with untreated controls.

FIGS. 7A-J. The effect of H₂O₂ on CE apoptosis and mitochondrial membrane potential ex vivo. Confocal images of whole mounts of corneal endothelium with detection of early apoptosis by annexin-V (green) (Ann+/PI−) and late apoptosis by annexin-V and propidium iodide (red) (Ann+/PI+). Low-dose H₂O₂ (1 μM, 37° C.) induced early apoptosis after 60 minutes (B) and 90 minutes (C), and late apoptosis after 2 hours (D and E) as compared to controls (A). Concurrent changes in mitochondrial membrane potential as detected by MitoTracker stain (red) were present at 60 minutes (G), 90 minutes (H), and 2 hours after the treatment (I and J). Controls were incubated in DMEM only at 37° C. for 0-12 hours, and no significant changes were detected (A and F). Results shown in E and J are means±SD and are representative of four independent experiments; *P<0.05, compared with untreated controls.

FIG. 8. Diagram of the pathogenesis of FECD. Endogenous and exogenous oxidative stress combined with genetic factors and postmitotic arrest of CE may lead to corneal edema seen in FECD since it causes 1) oxidant-antioxidant imbalance, 2) oxidative mitochondrial DNA damage, 3) apoptosis, and 4) CE morphological changes.

FIG. 9. Nrf2 is a key regulator of coordinated upregulation of antioxidant defense. In an oxidized state, phosphorylation of Nrf2 by kinases results in release of Nrf2 from Keap1, cytoplasmic stabilization and nuclear translocation of Nrf2. Keap1 targets Nrf2 for degradation and inhibits its activity. DJ-1 is a cytoplasmic stabilizer that aids in nuclear accumulation of Nrf2. In the nucleus, Nrf2 binds a common promoter region of multiple antioxidant defense enzymes and causes transcriptional activation of genes such as those we found underexpressed in FECD endothelium.

FIG. 10 is a photograph of an electrophoretic gel.

FIG. 11A is a bar graph showing that endothelial cell apoptosis in FECD was decreased by 58% p=1.57E-06 with SF treatment (without pro-oxidant conditions [tBHP-]). SF decreased endothelial cell apoptosis by as much as 45% (p=0.016) in specimens exposed to pro-oxidants or tBHP.

FIG. 11B is a bar graph showing that treatment with D3T decreased the percentage of apoptotic cells in FECD in the absence of oxidative stress.

### DETAILED DESCRIPTION OF THE INVENTION

Corneal endothelium (CE) is a monolayer of cells situated in the anterior chamber surface of the cornea; its primary function is to maintain the cornea in a state of deturgescence through sodium-activated ATPase pumping of water, thus, transparency. FECD is the most common cause of endogenous corneal endothelial degeneration and is characterized by alterations in corneal endothelial cell morphology, progressive loss of CE cells, and concomitant accumulation of extracellular deposits in the basement membrane that eventually lead to corneal edema and opacity.

Because CE cells do not divide in vivo, loss of endothelial cells seen in FECD is permanent. Prior to the invention, corneal transplantation has been the only treatment modality that can restore lost vision-rendering FECD the second most common cause of corneal transplants performed on the elderly (>60 years old) in the U.S. Lack of knowledge of the mechanism of CE degeneration in FECD has precluded the development of pharmacotherapeutics for this common and blinding condition.

FECD has been termed a disorder of aging; it is a bilateral and slowly progressive disorder, typically appearing after the age of 60. FECD is usually a sporadic condition, but it can be inherited as an autosomal dominant trait. FECD is characterized by endothelial cell apoptosis, endothelial cell morphological changes, and concomitant extracellular matrix deposition in the form of mound-shaped excrescences, termed guttae. The loss of CE cells and the formation of guttae start in the central cornea and spread toward the periphery. The number of endothelial cells remaining in the cornea is inversely proportional to the number of guttae excrescences. As the disease progresses, endothelial cell loss is accompanied by the thinning, stretching, and enlargement of neighboring CE cells as well as the loss of their hexagonal shape. Clinically, the endothelial morphological changes in FECD are denoted polymegethism, a variation in cell size, and pleomorphism, a variation in cell shape.

CE may be prone to oxidative stress due to its lifelong exposure to light (the cornea is in the direct light path to the retina), high oxygen demand from exuberant metabolic activity (it has to continually pump ions by Na+K+ATPases), and postmitotic arrest. Proteomic analysis of corneal endothelium taken from patients with FECD and age-matched normal controls has revealed decreased expression of peroxiredoxins (PRDXs), thioredoxin-dependent antioxidants that convert hydrogen peroxide (H₂O₂) to water. In addition, increased levels of advanced glycation end products, non enzymatically glycated proteins known to be associated with increased cellular oxidative stress, and their receptors, have been detected in FECD CE and Descemet's membrane compared to normal controls.

Pseudophakic bullous keratopathy (PBK) is the most common cause of exogenous loss of corneal endothelium such as seen during any intraocular ophthalmic surgery. Major surgeries that can affect the health of corneal endothelium and impair endothelial pumping and barrier function, are cataract surgery, glaucoma surgery, and retinal surgeries. During these surgeries, there is placement and manipulation of instruments inside of the eye as well as irrigation of the inside of the eye with intraocular solutions. Specifically, during cataract surgery, phacoemulcification energy is used to fragment the cataractous lens and that has been associated with oxidative stress on corneal endothelium. Phacoemulcification cataract surgery is one of the most common causes of corneal endothelial cell loss in PBK requiring corneal transplantation. Therefore, addition of antioxidant additives to already utilized solutions might be a very useful way to arrest the endothelial cell death pre and post intraocular surgery.

The foregoing description of related art is not intended in any way as an admission that any of the documents described therein, including pending United States patent applications, are prior art to embodiments of the present disclosure. Moreover, the description herein of any disadvantages associated with the described products, methods, and/or apparatus, is not intended to limit the disclosed embodiments. Indeed, embodiments of the present disclosure may include certain features of the described products, methods, and/or apparatus without suffering from their described disadvantages.

In the following description of the preferred embodiment, reference is made to the accompanying drawings which form a part hereof, and in which is shown by way of illustration a specific embodiment in which the invention may be practiced. It is to be understood that other embodiments may be utilized and structural changes may be made without departing from the scope of the present invention.

The present invention relates to activating binding of nuclear factor E2-related factor 2 (Nrf2) to the antioxidant response element (ARE) in methods for treating diseased or damaged corneal endothelium. Compositions comprising Nrf2 activators are used in the treatment the corneal endothelium, e.g., by reversing oxidant-antioxidant imbalance.

### Nrf2 Activators

Nrf2 activators are the primary active agent in the formulations described herein. Nrf2 activators include molecules that enhance Nrf2 levels or alleviate its degradation, which include, but are not limited to, the following: 1) hydroquinones, 2) sulforaphane, 3) polyphenols (such as reservatol), 4) curcumin, 5) catecols, 6) statins, combinations thereof, or a pharmacologically active derivative or analog thereof. In some embodiments, agents that downregulate Nrf2 antagonists are the primary active agent in the formulations of the present invention.

Nrf2 activating agents having stimulatory activity for Nrf2 protein nuclear translocation include, for example: Michael addition acceptors (e.g., α,β-unsaturated carbonyl compounds), such as diethyl maleate or dimethylfumarate; diphenols such as resveratrol, butylated hydroxyanisoles such as 2(3)-tert-butyl-4-hydroxyanisole, thiocarbamates such as pyrrolidinedithiocarbamate, quinones such as tert-butyl-hydroquinone, isothiocyanates such as sulforaphane, its precursor glucosinolate, glucoraphanin, or phenethyl isothiocyanate (PEITC), 1,2-dithiole-3-thiones such as oltipraz, 3,5-di-tert-butyl-4-hydroxytoluene, ethoxyquin, coumarins such as 3-hydroxycoumarin, flavonoids such as quercetin or curcumin, diallyl sulfide, indole-3-carbinol, epigallo-3-catechin gallate, ellagic acid, combinations thereof, or a pharmacologically active derivative or analog thereof.

Preferred agents having stimulatory activity for Nrf2 protein nuclear translocation include Butylated hydroxyanisole; 2,tert-butyl-hydroquinone; 3.ethoxyquin; pyrrolidin-edithiocarbamate; 3-hydroxycoumarin; sulforaphane; diethyl maleate; phorbol 12-myristate; phorbol 13-acetate; beta-naphthoflavone, and Oltipaz.

Examples of the Nrf2 activators include tert-butylhydroquinone, diethylmaleate sulforaphane, avicins, 15dPGJ₂, xanthohumol, curcumin, carnosol, zerumbone, isothiocyanate, α-lipoic acid, oltipraz (4-methyl-5-[2-pyrazinyl]-1,2-dithiole-3-thione), 1,2-dithiole-3-thione, and 2,3-butyl-4-hydroxyanisole, a Michael Addition acceptor, diphenol, thiocarbamate, quinone, butylated hydroxyanisole, flavonoid, an isothiocyanate, 3,5-di-tert-butyl-4-hydroxytoluene, ethoxyquin, combinations thereof, or a pharmacologically active derivative or analog thereof.

Other Nrf2 activators involve an isothiocyanate such as sulforaphane, or a pharmacologically active derivative thereof. In another embodiment, the agent comprises a 1,2-dithiole-3-thione such as oltipraz, or a pharmacologically active derivative thereof. Sulforaphane analogs include, for example, 6-(isothiocyanato-2-hexanone), exo-2-acetyl-6-isothiocyanatonorbornane, exo-2-(isothiocyanato-6-methylsu-lfonylnorbornane), 6-isothiocyanato-2-hexanol, 1-(isothiocyanato-4-dimethy-1phosphonylbutane, exo-2-(1-hydroxyethyl)-5-isothiocyanatonorbornane, exo-2-acetyl-5-isothiocyanatonorbornane, 1-(isothiocyanato-5-methylsulfon-ylpentane), cis-3-(methylsulfonyl)(cyclohexylmethylisothiocyanate) and trans-3-(methylsulfonyl)(cyclohexylmethylisothiocyanate).

Nrf2 activators also include dithiolethiones, which are a class of organosulfur compounds. Representative dithiolethiones include, but are not limited to, oltipraz.

A series of synthetic oleane triterpenoid compounds that are Nrf2 activators and referred to as Antioxidant Inflammatory Modulators (AIMs). Representative AIMs include, but are not limited to, bardoxolone methyl (also known as CDDO-Me or RTA 402).

Examples of polyphenols include, but are not limited to, isohumulones and related compounds, which include isohumulone, isohumulone, isohumulone, isoposthumulone, isoprehumulone, tetrahydroisohumulone, alloisohumulone, paraisohumulone, humulinic acid, hexahydroisohumulone, antiisohumulone, and humulones. In some embodiments, the polyphenol may be polyphenol antioxidants, which include, but are not limited to, the following: chlorogenic acid agent, fisetin, baicalein, and any combination thereof.

A Nrf2 activator and/or mitochondrial protective agent (e.g., mitochondrially targeted antioxidants) is useful in the methods and compositions of the present embodiments, e.g., N-acetylcysteine, indomethacin, glucobrassicin, glucoraphanin, ascorbigen, and indole-3-carbinole. Optionally, the compositions of the invention are administered with other detoxifying agents such as dimethyl caprol, glutathione, methionine, sodium hydrogen carbonate, deferoxamine mesylate, calcium disodium edetae, trientine hydrochloride, penicillamine, and pharmaceutical charcoal.

Neurite outgrowth-promoting prostaglandins (NEPP) may also be used in the compositions and methods. See e.g., Satoh T et al. Activation of the Keap1/Nrf2 pathway for neuroprotection by electrophilic phase II induceres. PNAS, 2006; 103:768-773, incorporated herein by reference in its entirety. Exemplary compounds include 13,14-dihydro-15-epi-Delta(7)-prostaglandin A(1) (methyl ester) and derivatives thereof.

Nrf2 activators include agents that activate Nrf2 and/or upstream or downstream regulators of Nrf2. Regulators of Nrf2 include, but are not limited to, the following: DJ-1 (PARK7) or Kelch-like ECH-associated protein 1 (Keap1).

Upon formulation, one or more kinds of medically effective active ingredients other than an active ingredient according to the present invention can be further admixed. Further, upon administering an active ingredient according to the present invention, one or more kinds of medically effective active ingredient other than an active ingredient according to the present invention can be combined for administration. Examples of such other active ingredients include, but are not limited to, Nrf2 activators, antioxidants, detoxification agents, and anti-inflammatory agents.

Examples of the Nrf2 activators are described in U.S. Publication No. 2007/0248705 A1 (incorporated herein by reference) and include sulforaphane, avicins, 15dPGJ2, xanthohumol, curcumin, carnosol, zerumbone, isothiocyanate, α-lipoic acid, oltipraz (4-methyl-5-[2-pyrazinyl]-1,2-dithiole-3-thione), 1,2-dithiole-3-thione, and 2,3-butyl-4-hydroxyanisole. Examples of the antioxidants include vitamin C, vitamin E, carotenoids, retinoids, polyphenols, flavonoids, lignan, selenium, butylated hydroxyanisole, ethylene diamine tetra-acetate, calcium disodium, acetylcysteine, probucol, and tempo). Examples of the detoxification agents include dimethyl caprol, glutathione, acetylcysteine, methionine, sodium hydrogen carbonate, deferoxamine mesylate, calcium disodium edetate, trientine hydrochloride, penicillamine, and pharmaceutical charcoal. The anti-inflammatory agents include steroidal anti-inflammatory agents and non-steroidal anti-inflammatory agents. Examples of the steroidal anti-inflammatory agents include cortisone acetate, hydrocortisone, paramethasone acetate, prednisolone, prednisolone, methylprednine, dexamethasone, triamcinolone, and betamethasone. Examples of the non-steroidal anti-inflammatory agents include salicylic acid non-steroidal anti-inflammatory agents such as aspirin, difiunisal, aspirin+ascorbic acid, and aspirin dialuminate; aryl acid non-steroidal anti-inflammatory agents such as diclofenac sodium, sulindac, fenbufen, indomethacin, indomethacin farnesyl, acemetacin, proglumetacin maleate, anfenac sodium, nabmeton, mofezolac, and etodorag; fenamic acid non-steroidal anti-inflammatory agents such as mefenamic acid, flufenamic acid aluminum, tolfenamic acid, and floctafenine; propionic acid non-steroidal anti-inflammatory agents such as ibuprofen, flurbiprofen, ketoprofen, naproxen, pranoprofen, fenoprofen calcium, thiaprofen, oxaprozin, loxoprofen sodium, alminoprofen, and zaltoprofen; oxicam non-steroldal anti-inflammatory agents such as piroxicam, ampiroxicam, tenoxicam, lornoxicam, and meloxicam; and basic non-steroidal anti-inflammatory agents such as tiaramide hydrochloride, epirizole, and emorfazone.

Agents that induce the Keap1-Nrf2 pathway (e.g., blocking Keap1-dependent Nrf2 ubiquitination/degradation and promoting stabilization and nuclear translocation of Nrf2 and subsequent induction of Nrf2 target genes) include antioxidant inflammation modulators (AIMS) such as synthetic oleane triterpenoid compounds (Reata Pharmaceuticals, Inc.). See U.S. Publication No. 20090326063, incorporated herein by reference in its entirety. AIMS include, but are not limited to, bardoxolone methyl (CDDO-Me or RTA 402). Methods for identifying agents that activates the Keap1-Nrf2-ARE pathway are disclosed in U.S. Publication No. 2010/0029012, incorporated herein by reference in its entirety.

Exemplary AIMs have the following formula:

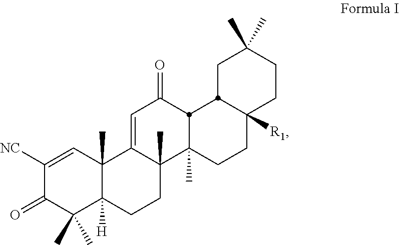

wherein R₁ is: —CN, or C₁-C₁₅-acyl or C₁-C₁₅-alkyl, wherein either of these groups is heteroatom-substituted or heteroatom-unsubstituted; or a pharmaceutically acceptable salt, hydrate or solvate thereof.

In some embodiments, the compound is defined as:

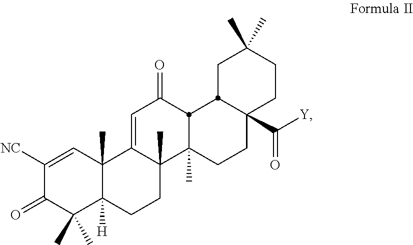

wherein Y is: —H, hydroxy, amino, halo, or C₁-C₁₄-alkoxy, C₂-C₁₄-alkenyloxy, C₂-C₁₄-alkynyloxy, C₁-C₁₄-aryloxy, C₂-C₁₄-aralkoxy, C₁-C₁₄-alkylamino, C₂-C₁₄-alkenylamino, C₂-C₁₄-alkynylamino, C₁-C₁₄-arylamino, C₃-C₁₀-aryl, or C₂-C₁₄-aralkylamino, wherein any of these groups is heteroatom-substituted or heteroatom-unsubstituted; or a pharmaceutically acceptable salt, hydrate or solvate thereof.

In some embodiments, Y is a heteroatom-unsubstituted C₁-C₄-alkylamino, such that the compound of the invention is, for example:

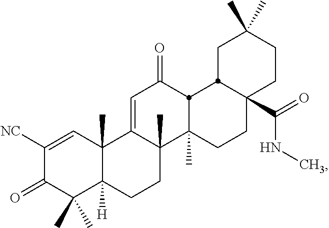

In some embodiments, Y is a heteroatom-substituted or heteroatom-unsubstituted C₂-C₄-alkylamino, such that the compound is, for example:

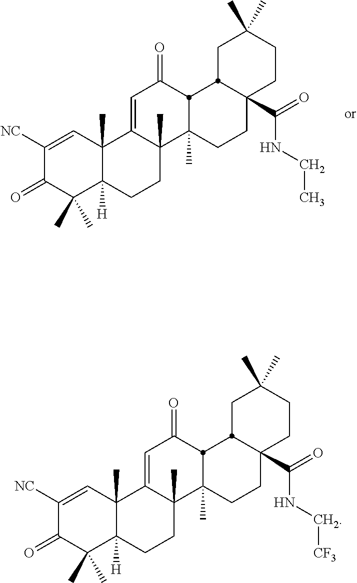

In some embodiments, Y is a heteroatom-substituted or heteroatom-unsubstituted C₁-C₄-alkoxy, such as a heteroatom-unsubstituted C₁-C₂-alkoxy. For example, one non-limiting example of such a compound is:

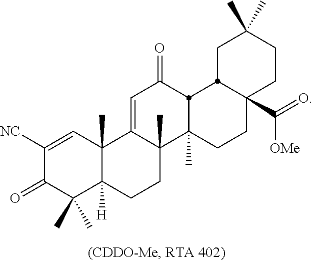

In some embodiments, at least a portion of the CDDO-Me is present as a polymorphic form, wherein the polymorphic form is a crystalline form having an X-ray diffraction pattern (CuKα) comprising significant diffraction peaks at about 8.8, 12.9, 13.4, 14.2 and 17.4a 20. In other variations, at least a portion of the CDDO-Me is present as a polymorphic form, wherein the polymorphic form is an amorphous form having an X-ray diffraction pattern (CuKα) with a halo peak at approximately 13.5a 20 and a T_{g}. In some variations, the compound is an amorphous form. In some variations, the compound is a glassy solid form of CDDO-Me, having an X-ray powder diffraction pattern with a halo peak at about 13.5α 2θ and a T_{g}. In some variations, the T_{g} value falls within a range of about 120α C. to about 135α C. In some variations, the T_{g} value is from about 125α C. to about 130α C.

In some embodiments, Y is hydroxy, such that the compound is, for example:

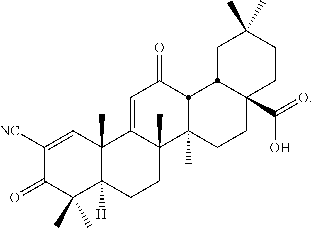

In some embodiments, the compound is:

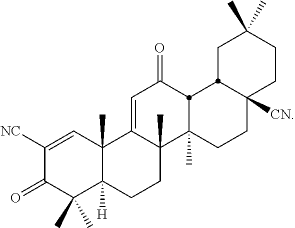

In some embodiments, the compound is defined as:

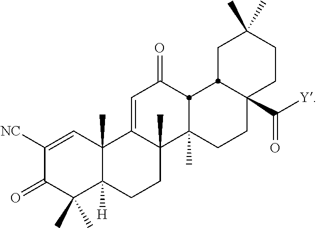

wherein Y′ is a heteroatom-substituted or heteroatom-unsubstituted C₁-C₁₄-aryl; or a pharmaceutically acceptable salt, hydrate or solvate thereof.

In some embodiments, the compound is:

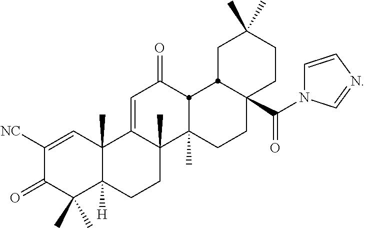

### Mitochondrial Targeted Antioxidants

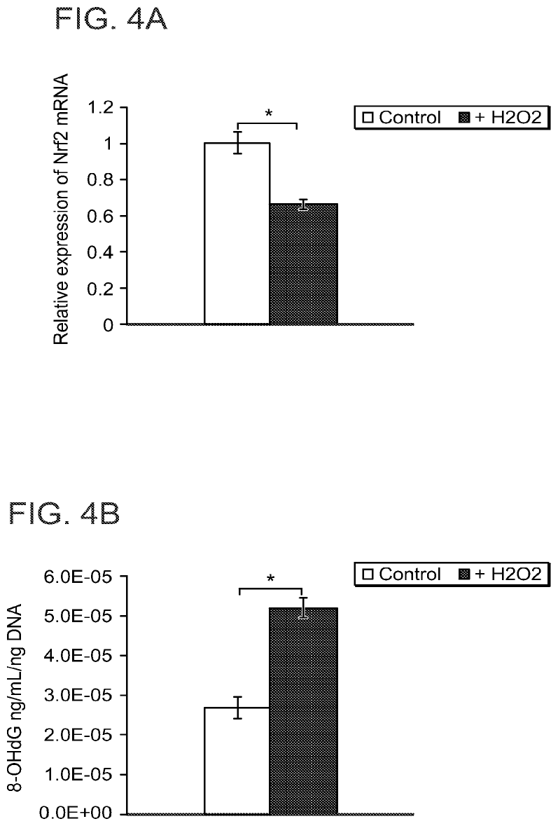

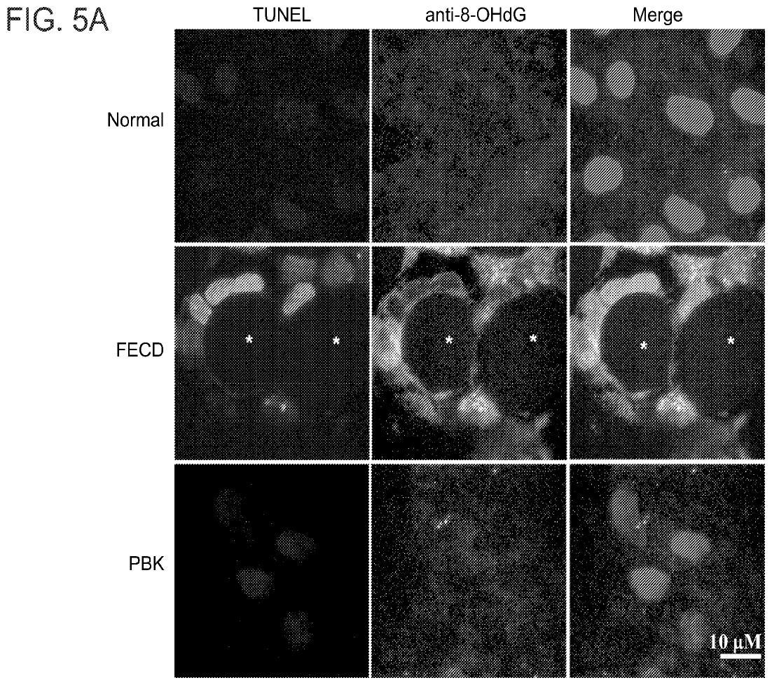

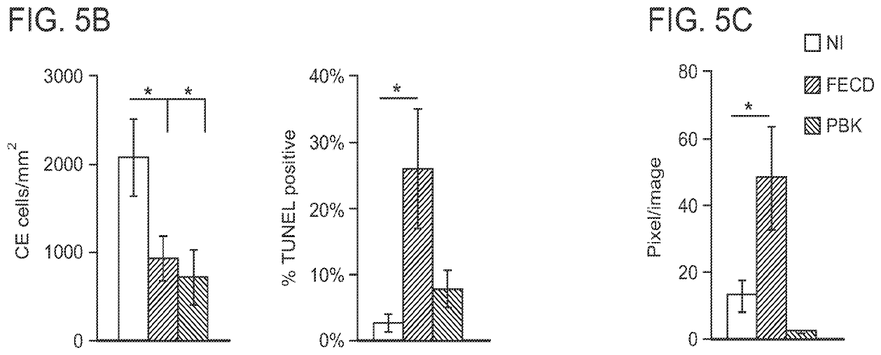

Detection of specific oxidative DNA damage of mitochondria in FECD is shown in FIG. 4. To the inventor's knowledge, these studies are first to show that mitochondria are specifically affected by oxidative damage in FECD, and that such damage is not present in other disorders that cause endothelial cell loss, such as pseudophakic bullous keratopathy (PBK) (FIG. 5). Mitochondria are the power houses of cells and are main organelles responsible for cellular energy production. Mitochondrial DNA mutations (as shown in FECD cells) accumulate progressively during life and result in deficiency of proteins necessary for proper functioning of the electron transport chain (ETC) that causes increased generation of reactive oxygen species (ROS). As a result, enhanced ROS production causes a ‘vicious cycle’ of increasing mitochondrial oxidative damage and degeneration.

The fact that mitochondrial DNA is present in FECD points to the specific therapeutics that can break the ‘vicious cycles’ of oxidation-induced degenerative changes. Mitochondrially targeted antioxidants that treat such DNA damage are 1) Szeto-Schiller (S-S) peptides, 2) MitoQ, 3) MitoVitE. Preferred mitochondrially targeted antioxidants include, but are not limited to Szeto-Schiller peptides (Szeto. Aaps. J. 8, 2006:E277-E283); TPP (Kelso et al. J. Biol. Chem. 280, 2005: 21295-21312); amd XJB-5-131 (Wipf et al. J. Am. Chem Soc. 127, 2005; 12460-12461, each of which is hereby incorporated by reference).

Mitochondrially targeted antioxidants refer to compounds that are rapidly and selectively accumulated by mitochondria. The targeting of vitamin E and coenzyme Q10 derivatives (U.S. Pat. No. 6,331,532; WO 99/26954; EP 1047701; WO2005/016322 and WO2005/016323) or a glutathione peroxidase mimetic (WO 2004/014927) to mitochondria by linking them to the triphenyl phosphonium ion has been described. These references are incorporated by references in their entireties. MitoQ is an orally active antioxidant that has the ability to target mitochondrial dysfunction.

Mitochondrially targeted antioxidants include, but are not limited to [2-(3,4-dihydro-6-hydroxy-2,5,7,8-tetramethyl-2H-1-benzopyran-2-yl)ethyl]-triphenylphosphonium bromide (MitoVit E), MitoQuinol [10-(3,6-dihydroxy-4,5-dimethoxy-2-methylphenyl)decyl]triphenylphosphonium bromide, and MitoQuinone [10-(4,5-dimethoxy-2-methyl-3,6-dioxo-1,4-cyclohexadien-1-yl)decyl]triphenylphosphonium bromide (MitoQ) or a MitoQ compound wherein the anion is methanesulfonate (James A. M. et al., 2005, J. Biol. Chem, 280: 21295-21312; WO2005/016322 and WO2005/016323). In a particularly preferred embodiment, the mitochondrially targeted antioxidants are MitoQuinol, or an oxidized form of the compound (wherein the hydroquinone of the formula is a quinone) referred to herein as “MitoQuinone”. A mixture of varying amounts of MitoQuinol and MitoQuinone is referred to as “MitoQ”. Even more preferably, the mitochondrially targeted antioxidant contains the pharmaceutically acceptable anion methanesulfonate. In this embodiment a mixture of varying amounts of MitoQuinol and MitoQuinone is referred to as “MitoS”.

Another mitochondrially targeted antioxidant is a glutathione peroxidase mimetic such as a selenoorganic compound, i.e. an organic compound comprising at least one selenium atom. Preferred classes of selenoorganic glutathione peroxidase mimetics include benzisoselenazolones, diaryl diselenides and diaryl selenides. In particular the glutathione peroxidase mimetic moiety is referred to herein as Ebelsen” (2-phenyl-benzo[d]isoselenazol-3-one). Additional mitochondrially targeted antioxidants are described in U.S. 2007/0161609, the entire content of which is incorporated herein by reference.

In some embodiments, the mitochondrially targeted antioxidant is one or more of the following: N-acetylcysteine, lipoic acid, melatonin, or resveratrol. See e.g., Zahid et al. Chem. Res. Toxicol. 20:1947-1953 (2007), incorporated by reference herein in its entirety.

### Eye Drops

The use of Nrf2 activators and/or mitochondrial protective agents (e.g., mitochondrially targeted antioxidants) in an eyedrop formulation for corneal endothelium enhances their effect by overcoming the bioavailability issue seen in systemic administration.

The eye drop is optionally formulated with or without one or more tear substitutes. In some embodiments, pharmaceutical compositions comprising an effective amount of one or more (e.g., 1, 2, 3, 4, 5, 6, 7, 8, 9, etc.) Nrf2 activators and a tear substitute are formulated in a pharmaceutically acceptable carrier for the treatment of the corneal endothelium. The Nrf2 activator and tear substitute act synergistically to provide a longer dwell time of the Nrf2 activator on the ocular surface, thus increasing duration and efficacy of action.

A variety of tear substitutes are known in the art and include, but are not limited to: monomeric polyols, such as, glycerol, propylene glycol, and ethylene glycol; polymeric polyols such as polyethylene glycol; cellulose esters such hydroxypropylmethyl cellulose, carboxy methylcellulose sodium and hydroxy propylcellulose; dextrans such as dextran 70; water soluble proteins such as gelatin; vinyl polymers, such as polyvinyl alcohol, polyvinylpyrrolidone, and povidone; and carbomers, such as carbomer 934P, carbomer 941, carbomer 940 and carbomer 974P. Many such tear substitutes are commercially available, which include, but are not limited to cellulose esters such as Bion Tears®, Celluvisc®, Genteal®, OccuCoat®, Refresh®, Teargen II®, Tears Naturale®, Tears Natural II®, Tears Naturale Free®, and TheraTears®; and polyvinyl alcohols such as Akwa Tears®, HypoTears®, Moisture Eyes®, Murine Lubricating®, and Visine Tears®. Tear substitutes may also be comprised of paraffins, such as the commercially available Lacri-Lube® ointments. Other commercially available ointments that are used as tear substitutes include Lubrifresh PM®, Moisture Eyes PM® and Refresh PM®.

Exemplary tear substitute contains hydroxypropylmethylcellulose. In certain embodiments, the tear substitute is Genteal® lubricating eye drops. GenTeal® (CibaVision-Novartis) is a sterile lubricant eye drop containing hydroxypropyl methylcellulose 3 mg/g and preserved with sodium perborate. Thus, the pharmaceutical compositions of the invention may comprise combinations of one or more Nrf2 activators, one or more mitochondrially targeted antioxidants and one or more tear substitutes. For example, the pharmaceutical compositions of the invention may comprise combinations of at least two Nrf2 activators and a tear substitute. In certain embodiments, the pharmaceutical compositions of the invention may comprise combinations of at least three Nrf2 activators and a tear substitute. In certain embodiments, the pharmaceutical compositions of the invention may comprise combinations of at least four Nrf2 activators and a tear substitute. In other embodiments, the topical formulations of the invention may comprise a Nrf2 activator and a combination of at least two tear substitutes. In other embodiments, the topical formulations of the invention may comprise a Nrf2 activator and a combination of at least three tear substitutes. In other embodiments, the topical formulations of the invention may comprise a Nrf2 activator and a combination of at least four tear substitutes. The pharmaceutical compositions of the invention may comprise combinations of at least two mitochondrially targeted antioxidants and a tear substitute. The pharmaceutical compositions of the invention may also comprise combinations of at least three mitochondrially targeted antioxidants and a tear substitute. In certain embodiments, the pharmaceutical compositions of the invention may comprise combinations of at least four mitochondrially targeted antioxidants and a tear substitute. In other embodiments, the topical formulations of the invention may comprise a mitochondrially targeted antioxidant and a combination of at least two tear substitutes. In other embodiments, the topical formulations of the invention may comprise an mitochondrially targeted antioxidant and a combination of at least three tear substitutes. In other embodiments, the topical formulations of the invention may comprise an mitochondrially targeted antioxidant and a combination of at least four tear substitutes.

### Nrf2-Antioxidant Response Element (ARE)-Driven Antioxidants

Nuclear factor E2-related factor 2 (Nrf2)-antioxidant response element (ARE) driven antioxidants are useful for treating the corneal endothelium. Nrf2-ARE driven antioxidants include, but are not limited to, gene products whose expression are induced by Nrf2. Nrf2 controls the inducible expression of numerous antioxidants and phase 2 detoxification genes, such as NAD(P)H quinone oxidoreductase 1 (NQO1), Glutamate-cysteine ligase, Heme oxygenase-1 (HMOX1), glutathione S-transferase (GST), UDP-glucuronosyltransferases (UGT), and Multidrug resistance-associated proteins (MRPs). Nrf2-ARE driven antioxidants may be topically administered alone or in combination with Nrf2 activators and/or mitochondrially targeted antioxidants according to the formulations and methods disclosed herein. In some embodiments, protein mimetics or protease resistant derivatives of Nrf2-ARE driven antioxidants are used.

Local delivery of Nrf2-ARE driven antioxidants to specific regions of the eye is carried out for example by gene therapy. For in vivo gene therapy, delivery may be systemic or local. Within the eye, gene therapy vector may be administered to the vitreous, the subretinal space and to the sub-tenar capsule.

A further important parameter is the dosage of Nrf2-ARE driven antioxidants to be delivered into the target tissue. In this regard, “unit dosage” refers generally to the concentration of Nrf2-ARE driven antioxidants/ml of Nrf2-ARE driven antioxidants composition. For viral vectors, the Nrf2-ARE driven antioxidants concentration is defined by the number of viral particles/ml of composition. Optimally, for delivery of Nrf2-ARE driven antioxidants using a viral expression vector, each unit dosage of Nrf2-ARE driven antioxidants will comprise 2.5 to 25 μL of a Nrf2-ARE driven antioxidants composition, wherein the composition includes a viral expression vector in a pharmaceutically acceptable fluid and provides from 1010 up to 1015 Nrf2-ARE driven antioxidants containing viral particles per ml of Nrf2-ARE driven antioxidants composition. Such high titers are particularly used for adeno-associated virus. For lentivirus, the titer is normally lower, such as from 108 to 1010 transducing units per ml (TU/ml).

The Nrf2-ARE driven antioxidant composition is delivered to each delivery cell site in the target tissue by microinjection, infusion, scrape loading, electroporation or other means suitable to directly deliver the composition directly into the delivery site. The delivery is accomplished slowly, such as over a period of about 5-10 minutes (depending on the total volume of Nrf2-ARE driven antioxidants composition to be delivered).

Viruses useful as gene transfer vectors include papovavirus, adenovirus, vaccinia virus, adeno-associated virus, herpesvirus, and retrovirus. Suitable retroviruses include HIV, SIV, FIV, EIAV, and/or MoMLV. Preferred viruses for treatment of disorders of the nervous system are lentiviruses and adeno-associated viruses. Both types of viruses can integrate into the genome without cell divisions, and both types have been tested in pre-clinical animal studies for indications of the nervous system, in particular the central nervous system. Methods for preparation of AAV are described in the art (e.g. U.S. Pat. No. 5,677,158. U.S. Pat. Nos. 6,309,634 and 6,683,058, the entire contents of which are incorporated by reference in their entireties) and provide examples of delivery of AAV to the central nervous system.

Special and preferred types of retroviruses include the lentiviruses which can transduce a cell and integrate into its genome without cell division. Thus preferably the vector is a replication-defective lentivirus particle. Such a lentivivus particle can be produced from a lentiviral vector comprising a 5′ lentiviral LTR, a tRNA binding site, a packaging signal, a promoter operably linked to a polynucleotide signal encoding said fusion protein, an origin of second strand DNA synthesis and a 3′ lentiviral LTR. Methods for preparation and in vivo administration of lentivirus to neural cells are described in US 2002/0037281, Methods for transducing neural cells using lentiviral vectors, the entire contents of which is incorporated by reference in their entirety.

Retroviral vectors are the vectors most commonly used in human clinical trials, since they carry a 7-8 kb which is more than many other viral vectors and since they have the ability to infect cells and have their genetic material stably integrated into the host cell with high efficiency. See, e.g., WO 95/30761; WO 95/24929, the entire contents of which are incorporated by reference in their entireties. Oncovirinae require at least one round of target cell proliferation for transfer and integration of exogenous nucleic acid sequences into the patient. Retroviral vectors integrate randomly into the patient's genome.

Two classes of retroviral particles have been described; ecotropic, which can infect mouse cells efficiently, and amphotropic, which can infect cells of many species. A third class includes xenotrophic retrovirus which can infect cells of another species than the species which produced the virus. Their ability to integrate only into the genome of dividing cells has made retroviruses attractive for marking cell lineages in developmental studies and for delivering therapeutic or suicide genes to cancers or tumors. These vectors may be particularly useful in the central nervous system, where there is a relative lack of cell division in adult patients.

For use in human patients, the retroviral vectors must be replication defective. This prevents further generation of infectious retroviral particles in the target tissue—instead the replication defective vector becomes a “captive” transgene stable incorporated into the target cell genome. Typically in replication defective vectors, the gag, env, and pol genes have been deleted (along with most of the rest of the viral genome). Heterologous DNA is inserted in place of the deleted viral genes. The heterologous genes may be under the control of the endogenous heterologous promoter, another heterologous promoter active in the target cell, or the retroviral 5′ LTR (the viral LTR is active in diverse tissues). Typically, retroviral vectors have a transgene capacity of about 7-8 kb.

Replication defective retroviral vectors require provision of the viral proteins necessary for replication and assembly in trans, from, e.g., engineered packaging cell lines. It is important that the packaging cells do not release replication competent virus and/or helper virus. This has been achieved by expressing viral proteins from RNAs lacking the w signal, and expressing the gag/pol genes and the env gene from separate transcriptional units. In addition, in some packaging cell lines, the 5′ LTR's have been replaced with non-viral promoters controlling expression of these genes and polyadenylation signals have been added. These designs minimize the possibility of recombination leading to production of replication competent vectors, or helper viruses. See, e.g., U.S. Pat. No. 4,861,719, herein incorporated by reference.

Construction of vectors for recombinant expression of Nrf2-ARE driven antioxidants for use in the invention may be accomplished using conventional techniques which do not require detailed explanation to one of ordinary skill in the art. Nrf2-ARE driven antioxidants produced by recombinant expression may be topically applied to the eye according the formulations and methods provided herein.

To form a Nrf2-ARE driven antioxidant composition for use in the invention, Nrf2-ARE driven antioxidants encoding expression viral vectors are placed into a pharmaceutically acceptable suspension, solution or emulsion. Suitable mediums include saline and liposomal preparations. More specifically, pharmaceutically acceptable carriers may include sterile aqueous of non-aqueous solutions, suspensions, and emulsions. Examples of nonaqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media.

Preservatives and other additives may also be present such as, for example, antimicrobials, antioxidants, chelating agents, and inert gases and the like. Further, a composition of Nrf2-ARE driven antioxidants transgenes may be lyophilized using means well known in the art, for subsequent reconstitution and use according to the invention. A colloidal dispersion system may also be used for targeted gene delivery. Colloidal dispersion systems include macromolecule complexes, nanocapsules, microspheres, beads, and lipid-based systems including oil-in-water emulsions, micelles, mixed micelles, and liposoms. Liposomes are artificial membrane vesicles which are useful as delivery vehicles in vitro and in vivo. It has been shown that large unilamellar vesicles (LUV), which range in size from 0.2-4.0 μm can encapsulate a substantial percentage of an aqueous buffer containing large macro molecules. RNA, DNA and intact virions can be encapsulated within the aqueous interior and be delivered to cells in a biologically active form. In addition to mammalian cells, liposomes have been used for delivery of operatively encoding transgenes in plant, yeast and bacterial cells. In order for a liposome to be an efficient gene transfer vehicle, the following characteristics should be present: (1) encapsulation of the genes encoding the Nrf2-ARE driven antioxidants at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information.

The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and di stearoylphosphatidylcholine.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs which contain sinusoidal capillaries.

Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted gene delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand.

A further example of a delivery system includes transplantation into the therapeutic area of a composition of packaging cells capable of producing vector particles as described in the present invention. Methods for encapsulation and transplantation of such cells are known in the art, in particular from WO 97/44065 (Cytotherapeutics).

By selecting a packaging cell line capable of producing lentiviral particles, transduction of non-dividing cells in the therapeutic area is obtained. By using retroviral particles capable of transducing only dividing cells, transduction is restricted to de-novo differentiated cells in the therapeutic area.

Direct delivery of a Nrf2-ARE driven antioxidants may be achieved by means familiar to those of skill in the art, including microinjection through a surgical incision; electropotation; infusion, chemical complexation with a targeting molecule or co-precipitant (e.g., liposome, calcium), and microparticle bombardment of the target tissue.

### Pharmaceutical Compositions

Compositions within the scope of the present invention contain the active agent(s) in an amount effective to achieve the desired therapeutic effect (e.g., prevention or reduction of endothelial cell death or loss from disease or as a consequence of intraocular surgery) while avoiding adverse side effects. Pharmaceutically acceptable preparations and salts of the active agent are within the scope of the present invention and are well known in the art. The amount of the therapeutic or pharmaceutical composition which is effective in the treatment of a particular disease, disorder or condition depends on the nature and severity of the disease, the target site of action, the patient's weight, special diets being followed by the patient, concurrent medications being used, the administration route and other factors that will be recognized by those skilled in the art. The dosage is adapted by the clinician in accordance with conventional factors such as the extent of the disease and different parameters from the patient. Typically, 0.001 to 100 mg/kg/day will be administered to the subject. Effective doses may be extrapolated from dose response curves derived from in vitro or animal model test systems.

Various delivery systems are known and can be used to the active agent(s). The pharmaceutical composition of the present invention can be administered by any suitable route including, intravenous or intramuscular injection, intraventricular or intrathecal injection (for central nervous system administration), orally, topically, subcutaneously, subconjunctivally, or via intranasal, intradermal, sublingual, vaginal, rectal or epidural routes.

Other delivery systems well known in the art can be used for delivery of the pharmaceutical compositions of the present invention, for example via aqueous solutions, encapsulation in microparticles, or microcapsules.

In yet another embodiment, the pharmaceutical compositions of the present invention can be delivered in a controlled release system. In one embodiment polymeric materials can be used, in another embodiment, a pump may be used.

In some embodiments, the active agent(s) may be combined with a pharmaceutically acceptable carrier (e.g., antioxidants, wetting agents, buffers, and tonicity adjusters). The term carrier refers to diluents, adjuvants, excipients such as filler or a binder, a disintegrating agent, a lubricant a silica flow conditioner a stabilizing agent or vehicles with which the active agent(s) is administered. Such pharmaceutical carriers include sterile liquids such as water and oils including mineral oil, vegetable oil (e.g., peanut oil, soybean oil, sesame oil, canola oil), animal oil or oil of synthetic origin. Aqueous glycerol and dextrose solutions as well as saline solutions may also be employed as liquid carriers of the pharmaceutical compositions of the present invention. Of course, the choice of the carrier depends on the nature of the active agent(s), its solubility and other physiological properties as well as the target site of delivery and application. For example, carriers that can penetrate the blood brain barrier are used for treatment, prophylaxis or amelioration of symptoms of diseases or conditions (e.g. inflammation) in the central nervous system. Examples of suitable pharmaceutical carriers are described in Remington: The Science and Practice of Pharmacy by Alfonso R. Gennaro, 2003, 21st edition, Mack Publishing Company.

Further pharmaceutically suitable materials that may be incorporated in pharmaceutical preparations of the present invention include absorption enhancers, pH regulators and buffers, osmolarity adjusters, preservatives, stabilizers, antioxidants, surfactants, thickeners, emollient, dispersing agents, flavoring agents, coloring agents and wetting agents.

Examples of suitable pharmaceutical excipients include, water, glucose, sucrose, lactose, glycol, ethanol, glycerol monostearate, gelatin, rice, starch, flour, chalk, sodium stearate, malt, sodium chloride and the like. The pharmaceutical compositions of the present invention can take the form of solutions, capsules, tablets, creams, gels, powders, sustained release formulations and the like. The composition can be formulated as a suppository, with traditional binders and carriers such as triglycerides (see Remington: The Science and Practice of Pharmacy by Alfonso R. Gennaro, 2003, 21.sup.th edition, Mack Publishing Company). Such compositions contain a therapeutically effective amount of the therapeutic composition, together with a suitable amount of carrier so as to provide the form for proper administration to the subject. The formulations are designed so as to suit the mode of administration and the target site of action (e.g., a particular organ or cell type).

Examples of fillers or binders that may be used in accordance with the present invention include acacia, alginic acid, calcium phosphate (dibasic), carboxymethylcellulose, carboxymethylcellulose sodium, hydroxyethylcellulose, hydroxypropylcellulose, hydroxypropylmethylcellulose, dextrin, dextrates, sucrose, tylose, pregelatinized starch, calcium sulfate, amylose, glycine, bentonite, maltose, sorbitol, ethylcellulose, disodium hydrogen phosphate, disodium phosphate, disodium pyrosulfite, polyvinyl alcohol, gelatin, glucose, guar gum, liquid glucose, compressible sugar, magnesium aluminum silicate, maltodextrin, polyethylene oxide, polymethacrylates, povidone, sodium alginate, tragacanth, microcrystalline cellulose, starch, and zein. Another most preferred filler or binder consists of microcrystalline cellulose.

Examples of disintegrating agents that may be used include alginic acid, carboxymethylcellulose, carboxymethylcellulose sodium, hydroxypropylcellulose (low substituted), microcrystalline cellulose, powdered cellulose, colloidal silicon dioxide, sodium croscarmellose, crospovidone, methylcellulose, polacrilin potassium, povidone, sodium alginate, sodium starch glycolate, starch, disodium disulfite, disodium edathamil, disodium edetate, di sodiumethylenediaminetetraacetate (EDTA) crosslinked polyvinylpyrollidines, pregelatinized starch, carboxymethyl starch, sodium carboxymethyl starch and microcrystalline cellulose.

Examples of lubricants include calcium stearate, canola oil, glyceryl palmitostearate, hydrogenated vegetable oil (type I), magnesium oxide, magnesium stearate, mineral oil, poloxamer, polyethylene glycol, sodium lauryl sulfate, sodium stearate fumarate, stearic acid, talc, zinc stearate, glyceryl behapate, magnesium lauryl sulfate, boric acid, sodium benzoate, sodium acetate, sodium benzoate/sodium acetate (in combination) and DL leucine.

Examples of silica flow conditioners include colloidal silicon dioxide, magnesium aluminum silicate and guar gum. Another most preferred silica flow conditioner consists of silicon dioxide.

Examples of stabilizing agents include acacia, albumin, polyvinyl alcohol, alginic acid, bentonite, dicalcium phosphate, carboxymethylcellulose, hydroxypropylcellulose, colloidal silicon dioxide, cyclodextrins, glyceryl monostearate, hydroxypropyl methylcellulose, magnesium trisilicate, magnesium aluminum silicate, propylene glycol, propylene glycol alginate, sodium alginate, carnauba wax, xanthan gum, starch, stearate(s), stearic acid, stearic monoglyceride and stearyl alcohol.

In some cases, the pharmaceutical formulations of the present invention are formulated for oral delivery. According to some embodiments, the pharmaceutical formulations of the present invention are formulated for ophthalmic delivery. In some embodiments, the pharmaceutical compositions are formulated for subconjunctival administration. In some embodiments, the pharmaceutical compositions are formulated for topical administration to the eye or region of the eye. In some embodiments, the formulation may comprise one or more tear substitutes. In some embodiments, the formulation may comprise an ophthalmic lubricant. The formulation the pH of the formulation is between 5.5 and 7. In some embodiments, the formulation is an aqueous formulation. In some embodiments, the formulation is in the form of a single dose unit. Ophthalmic formulations, eye ointments, creams, salves, powders, solutions and the like, are also contemplated as being within the scope of this invention.

Furthermore, various other delivery systems are known and can be used to administer the therapeutic agents of the present invention. In some embodiments, the pharmaceutical compositions may be administered by any suitable route including, orally, subcutaneously, parenterally, intravenously, local injection, intraocular injection, subconjunctivally, intranasal, intradermal, and sublingual. The pharmaceutical compositions may be administered by retrobulbar, intravitreal, intraretinal, or subconjunctival injection.

The formulation may be also administered as a slow release formulation, with a carrier formulation such as microspheres, microcapsules, liposomes, etc., as a topical ointment or solution, an intravenous solution or suspension, or in an intraocular injection, as known to one skilled in the art to treat or prevent an ophthalmic condition. Microsphere delivery systems or sustained-release delivery systems suitable for ocular use or delivery, and related compositions and methods, are disclosed in U.S. Pat. Nos. 5,837,226, 5,731,005, U.S.

Publication No. 2008/0131484, U.S. Pat. Nos. 5,641,750, and 7,354,574, each of which are incorporated herein by reference in its entirety. The microspheres may be suspended in a liquid solution which can be applied conveniently to the eye.

In some embodiments, the composition is suitable for local administration to the retina. In some embodiments, the composition is administered by retrobulbar, intravitreal, intraretinal, or subconjunctival injection. In some embodiments, the composition further comprises one or more tear substitutes. In some embodiments, the composition further comprises an ophthalmic lubricant. In some embodiments, the subject is human.

### Ophthalmic Formulations

In certain embodiments, the pharmaceutical compositions of the invention may comprise combinations of at least one (e.g., 1, 2, 3, 4, 5, 6, etc.) Nrf2 activator. In certain embodiments, the pharmaceutical compositions of the invention may comprise combinations of at least one (e.g., 1, 2, 3, 4, 5, 6, etc.) mitochondrially targeted antioxidant. In certain embodiments, the pharmaceutical compositions of the invention may comprise combinations of at least one Nrf2 activator (e.g., 1, 2, 3, 4, 5, 6, etc.) and at least one mitochondrially targeted antioxidants. According to some embodiments, the pharmaceutical compositions are formulated for topical administration to the eye (e.g., eye drops). According to some embodiments, the pharmaceutical compositions may further comprise a tear substitute.

In certain embodiments, the concentration of Nrf2 activators are from 0.10% to 10.0% (w/v), including but not limited to, from 0.1% to 10%, from 0.1% to 9.5%, from 0.1% to 9%, from 0.1% to 8.5%, from 0.1% to 8%, from 0.1% to 7.5%, from 0.1% to 7%, from 0.1% to 10%, from 0.1% to 6%, from 0.1% to 5.5%, from 0.1% to 5%, from 0.1% to 4.5%, from 0.1% to 4%, from 0.1% to 3.5%, from 0.1% to 3%, from 0.1% to 2.5%, from 0.1% to 2%, from 0.1% to 1.5%, from 0.1% to 1%, from 0.1% to 0.5%, from 0.2% to 10%, from 0.2% to 9.5%, from 0.2% to 9%, from 0.2% to 8.5%, from 0.2% to 8%, from 0.2% to 7.5%, from 0.2% to 7%, from 0.2% to 10%, from 0.2% to 6%, from 0.2% to 5.5%, from 0.2% to 5%, from 0.2% to 4.5%, from 0.2% to 4%, from 0.2% to 3.5%, from 0.2% to 3%, from 0.2% to 2.5%, from 0.2% to 2%, from 0.2% to 1.5%, from 0.2% to 1%, from 0.2% to 0.5%, from 0.5% to 10%, from 0.5% to 9.5%, from 0.5% to 9%, from 0.5% to 8.5%, from 0.5% to 8%, from 0.5% to 7.5%, from 0.5% to 7%, from 0.5% to 10%, from 0.5% to 6%, from 0.5% to 5.5%, from 0.5% to 5%, from 0.5% to 4.5%, from 0.5% to 4%, from 0.5% to 3.5%, from 0.5% to 3%, from 0.5% to 2.5%, from 0.5% to 2%, from 0.5% to 1.5%, from 0.5% to 1%, from 1% to 10%, from 1% to 9.5%, from 1% to 9%, from 1% to 8.5%, from 1% to 8%, from 1% to 7.5%, from 1% to 7%, from 1% to 10%, from 1% to 6%, from 1% to 5.5%, from 1% to 5%, from 1% to 4.5%, from 1% to 4%, from 1% to 3.5%, from 1% to 3%, from 1% to 2.5%, from 1% to 2%, from 1% to 1.5%, from 2% to 10%, from 2% to 9.5%, from 2% to 9%, from 2% to 8.5%, from 2% to 8%, from 2% to 7.5%, from 2% to 7%, from 2% to 10%, from 2% to 6%, from 2% to 5.5%, from 2% to 5%, from 2% to 4.5%, from 2% to 4%, from 2% to 3.5%, from 2% to 3%, from 2% to 2.5%, from 3% to 10%, from 3% to 9.5%, from 3% to 9%, from 3% to 8.5%, from 3% to 8%, from 3% to 7.5%, from 3% to 7%, from 3% to 10%, from 3% to 6%, from 3% to 5.5%, from 3% to 5%, from 3% to 4.5%, from 3% to 4%, from 3% to 3.5%, from 4% to 10%, from 4% to 9.5%, from 4% to 9%, from 4% to 8.5%, from 4% to 8%, from 4% to 7.5%, from 4% to 7%, from 4% to 10%, from 4% to 6%, from 4% to 5.5%, from 4% to 5%, from 4% to 4.5%, from 5% to 10%, from 5% to 9.5%, from 5% to 9%, from 5% to 8.5%, from 5% to 8%, from 5% to 7.5%, from 5% to 7%, from 5% to 10%, from 5% to 6%, and from 5% to 5.5%.

The concentration of mitochondrially targeted antioxidants may be from 0.10% to 10.0% (w/v), including but not limited to, from 0.1% to 10%, from 0.1% to 9.5%, from 0.1% to 9%, from 0.1% to 8.5%, from 0.1% to 8%, from 0.1% to 7.5%, from 0.1% to 7%, from 0.1% to 10%, from 0.1% to 6%, from 0.1% to 5.5%, from 0.1% to 5%, from 0.1% to 4.5%, from 0.1% to 4%, from 0.1% to 3.5%, from 0.1% to 3%, from 0.1% to 2.5%, from 0.1% to 2%, from 0.1% to 1.5%, from 0.1% to 1%, from 0.1% to 0.5%, from 0.2% to 10%, from 0.2% to 9.5%, from 0.2% to 9%, from 0.2% to 8.5%, from 0.2% to 8%, from 0.2% to 7.5%, from 0.2% to 7%, from 0.2% to 10%, from 0.2% to 6%, from 0.2% to 5.5%, from 0.2% to 5%, from 0.2% to 4.5%, from 0.2% to 4%, from 0.2% to 3.5%, from 0.2% to 3%, from 0.2% to 2.5%, from 0.2% to 2%, from 0.2% to 1.5%, from 0.2% to 1%, from 0.2% to 0.5%, from 0.5% to 10%, from 0.5% to 9.5%, from 0.5% to 9%, from 0.5% to 8.5%, from 0.5% to 8%, from 0.5% to 7.5%, from 0.5% to 7%, from 0.5% to 10%, from 0.5% to 6%, from 0.5% to 5.5%, from 0.5% to 5%, from 0.5% to 4.5%, from 0.5% to 4%, from 0.5% to 3.5%, from 0.5% to 3%, from 0.5% to 2.5%, from 0.5% to 2%, from 0.5% to 1.5%, from 0.5% to 1%, from 1% to 10%, from 1% to 9.5%, from 1% to 9%, from 1% to 8.5%, from 1% to 8%, from 1% to 7.5%, from 1% to 7%, from 1% to 10%, from 1% to 6%, from 1% to 5.5%, from 1% to 5%, from 1% to 4.5%, from 1% to 4%, from 1% to 3.5%, from 1% to 3%, from 1% to 2.5%, from 1% to 2%, from 1% to 1.5%, from 2% to 10%, from 2% to 9.5%, from 2% to 9%, from 2% to 8.5%, from 2% to 8%, from 2% to 7.5%, from 2% to 7%, from 2% to 10%, from 2% to 6%, from 2% to 5.5%, from 2% to 5%, from 2% to 4.5%, from 2% to 4%, from 2% to 3.5%, from 2% to 3%, from 2% to 2.5%, from 3% to 10%, from 3% to 9.5%, from 3% to 9%, from 3% to 8.5%, from 3% to 8%, from 3% to 7.5%, from 3% to 7%, from 3% to 10%, from 3% to 6%, from 3% to 5.5%, from 3% to 5%, from 3% to 4.5%, from 3% to 4%, from 3% to 3.5%, from 4% to 10%, from 4% to 9.5%, from 4% to 9%, from 4% to 8.5%, from 4% to 8%, from 4% to 7.5%, from 4% to 7%, from 4% to 10%, from 4% to 6%, from 4% to 5.5%, from 4% to 5%, from 4% to 4.5%, from 5% to 10%, from 5% to 9.5%, from 5% to 9%, from 5% to 8.5%, from 5% to 8%, from 5% to 7.5%, from 5% to 7%, from 5% to 10%, from 5% to 6%, and from 5% to 5.5%.

Preferably, the pharmaceutical compositions according to the present invention are formulated as solutions, suspensions and other dosage forms for topical administration. Aqueous solutions are generally preferred, based on ease of formulation, as well as a patient's ability to easily administer such compositions by means of instilling one to two drops of the solutions in the affected eyes. However, the compositions may also be suspensions, viscous or semi-viscous gels, or other types of solid or semi-solid compositions.

Any of a variety of carriers may be used in the formulations of the present invention including water, mixtures of water and water-miscible solvents, such as C₁- to C₇-alkanols, vegetable oils or mineral oils comprising from 0.5 to 5% non-toxic water-soluble polymers, natural products, such as gelatin, alginates, pectins, tragacanth, karaya gum, xanthan gum, carrageenin, agar and acacia, starch derivatives, such as starch acetate and hydroxypropyl starch, and also other synthetic products, such as polyvinyl alcohol, polyvinylpyrrolidone, polyvinyl methyl ether, polyethylene oxide, preferably cross-linked polyacrylic acid, such as neutral Carbopol, or mixtures of those polymers. The concentration of the carrier is, typically, from 1 to 100000 times the concentration of the active ingredient. Additional ingredients that may be included in the formulation include tonicity enhancers, preservatives, solubilizers, non-toxic excipients, demulcents, sequestering agents, pH adjusting agents, co-solvents and viscosity building agents. For the adjustment of the pH, preferably to a physiological pH, buffers may especially be useful. The pH of the present solutions should be maintained within the range of 4.0 to 8.0, more preferably about 4.0 to 6.0, more preferably about 6.5 to 7.8. Suitable buffers may be added, such as boric acid, sodium borate, potassium citrate, citric acid, sodium bicarbonate, TRIS, and various mixed phosphate buffers (including combinations of Na2HPO4, NaH2PO4 and KH2PO4) and mixtures thereof. Borate buffers are preferred. Generally, buffers will be used in amounts ranging from about 0.05 to 10 percent by weight (e.g., from about 0.1% to about 1.5%, from about 0.05% to about 0.5%, from about 0.05% to about 1%, from about 0.05% to about 1.5%, from about 0.05% to about 2%, from about 0.05% to about 2.5%, from about 0.05% to about 2.5%, from about 0.05% to about 3%, from about 0.05% to about 5%, from about 0.1% to about 0.5%, from about 0.1% to about 1%, from about 0.1% to about 1.5%, from about 0.1% to about 2%, from about 0.1% to about 2.5%, from about 0.1% to about 2.5%, from about 0.1% to about 3%, from about 0.1% to about 5%, from about 0.2% to about 0.5%, from about 0.2% to about 1%, from about 0.2% to about 1.5%, from about 0.2% to about 2%, from about 0.2% to about 2.5%, from about 0.2% to about 2.5%, from about 0.2% to about 3%, from about 0.2% to about 5%, from about 0.5% to about 1%, from about 0.5% to about 1.5%, from about 0.5% to about 2%, from about 0.5% to about 2.5%, from about 0.5% to about 2.5%, from about 0.5% to about 3%, from about 0.5% to about 5%, from about 1% to about 1%, from about 1% to about 1.5%, from about 1% to about 2%, from about 1% to about 2.5%, from about 1% to about 2.5%, from about 1% to about 3%, or from about 1% to about 5%).

Tonicity is adjusted if needed by tonicity enhancing agents. Such agents may, for example be of ionic and/or non-ionic type. Examples of ionic tonicity enhancers are alkali metal or earth metal halides, such as, for example, CaCl₂), KBr, KCl, LiCl, Nal, NaBr or NaCl, Na₂SO₄ or boric acid. Non-ionic tonicity enhancing agents are, for example, urea, glycerol, sorbitol, mannitol, propylene glycol, or dextrose. The aqueous solutions of the present invention are typically adjusted with tonicity agents to approximate the osmotic pressure of normal lachrymal fluids which is equivalent to a 0.9%±0.1% solution of sodium chloride or a 2.5%±0.3% solution of glycerol. An osmolality of about 225 to 400 mOsm/kg is preferred, more preferably 280 to 320 mOsm.

In certain embodiments, the topical formulations additionally comprise a preservative. A preservative may typically be selected from a quaternary ammonium compound such as benzalkonium chloride, benzoxonium chloride or the like. Benzalkonium chloride is better described as: N-benzyl-N—(C₈-C₁₈ alkyl)-N,N-dimethylammonium chloride. Examples of preservatives different from quaternary ammonium salts are alkyl-mercury salts of thiosalicylic acid, such as, for example, thiomersal, phenylmercuric nitrate, phenylmercuric acetate or phenylmercuric borate, sodium perborate, sodium chlorite, parabens, such as, for example, methylparaben or propylparaben, alcohols, such as, for example, chlorobutanol, benzyl alcohol or phenyl ethanol, guanidine derivatives, such as, for example, chlorohexidine or polyhexamethylene biguanide, sodium perborate, Germal®II or sorbic acid. Preferred preservatives are quaternary ammonium compounds, in particular benzalkonium chloride or its derivative such as Polyquad (see U.S. Pat. No. 4,407,791), alkyl-mercury salts and parabens. Where appropriate, a sufficient amount of preservative is added to the ophthalmic composition to ensure protection against secondary contaminations during use caused by bacteria and fungi.

In another embodiment, the topical formulations of this invention do not include a preservative. Such formulations would be useful for patients who wear contact lenses, or those who use several topical ophthalmic drops and/or those with an already compromised ocular surface (e.g. dry eye) wherein limiting exposure to a preservative may be more desirable.

The topical formulation may additionally require the presence of a solubilizer, in particular if the active or the inactive ingredients tends to form a suspension or an emulsion. A solubilizer suitable for an above concerned composition is for example selected from the group consisting of tyloxapol, fatty acid glycerol polyethylene glycol esters, fatty acid polyethylene glycol esters, polyethylene glycols, glycerol ethers, a cyclodextrin (for example alpha-, beta- or gamma-cyclodextrin, e.g. alkylated, hydroxyalkylated, carboxyalkylated or alkyloxycarbonyl-alkylated derivatives, or mono- or diglycosyl-alpha-, beta- or gamma-cyclodextrin, mono- or dimaltosyl-alpha-, beta- or gamma-cyclodextrin or panosyl-cyclodextrin), polysorbate 20, polysorbate 80 or mixtures of those compounds. A specific example of an especially preferred solubilizer is a reaction product of castor oil and ethylene oxide, for example the commercial products Cremophor EL® or Cremophor RH40®. Reaction products of castor oil and ethylene oxide have proved to be particularly good solubilizers that are tolerated extremely well by the eye. Another preferred solubilizer is selected from tyloxapol and from a cyclodextrin. The concentration used depends especially on the concentration of the active ingredient. The amount added is typically sufficient to solubilize the active ingredient. For example, the concentration of the solubilizer is from 0.1 to 5000 times the concentration of the active ingredient. The formulations may comprise further non-toxic excipients, such as, for example, emulsifiers, wetting agents or fillers, such as, for example, the polyethylene glycols designated 200, 300, 400 and 600, or Carbowax designated 1000, 1500, 4000, 6000 and 10000. The amount and type of excipient added is in accordance with the particular requirements and is generally in the range of from approximately 0.0001 to approximately 90% by weight (e.g., from about 0.1% to about 5%, from about 0.1% to about 10%, from about 0.1% to about 15%, from about 0.1% to about 20%, from about 0.1% to about 25%, from about 0.1% to about 30%, from about 0.1% to about 35%, from about 0.1% to about 40%, from about 0.1% to about 45%, from about 0.1% to about 50%, from about 0.1% to about 55%, from about 0.1% to about 60%, from about 0.1% to about 65%, from about 0.1% to about 70%, from about 0.1% to about 75%, from about 0.1% to about 80%, from about 0.1% to about 85%, from about 0.1% to about 90%, from about 1% to about 5%, from about 1% to about 10%, from about 1% to about 15%, from about 1% to about 20%, from about 1% to about 25%, from about 1% to about 30%, from about 1% to about 35%, from about 1% to about 40%, from about 1% to about 45%, from about 1% to about 50%, from about 1% to about 55%, from about 1% to about 60%, from about 1% to about 65%, from about 1% to about 70%, from about 1% to about 75%, from about 1% to about 80%, from about 1% to about 85%, from about 1% to about 90%, from about 5% to about 10%, from about 5% to about 15%, from about 5% to about 20%, from about 5% to about 25%, from about 5% to about 30%, from about 5% to about 35%, from about 5% to about 40%, from about 5% to about 45%, from about 5% to about 50%, from about 5% to about 55%, from about 5% to about 60%, from about 5% to about 65%, from about 5% to about 70%, from about 5% to about 75%, from about 5% to about 80%, from about 5% to about 85%, from about 5% to about 90%, from about 10% to about 15%, from about 10% to about 20%, from about 10% to about 25%, from about 10% to about 30%, from about 10% to about 35%, from about 10% to about 40%, from about 10% to about 45%, from about 10% to about 50%, from about 10% to about 55%, from about 10% to about 60%, from about 10% to about 65%, from about 10% to about 70%, from about 10% to about 75%, from about 10% to about 80%, from about 10% to about 85%, from about 10% to about 90%, from about 20% to about 15%, from about 20% to about 20%, from about 20% to about 25%, from about 20% to about 30%, from about 20% to about 35%, from about 20% to about 40%, from about 20% to about 45%, from about 20% to about 50%, from about 20% to about 55%, from about 20% to about 60%, from about 20% to about 65%, from about 20% to about 70%, from about 20% to about 75%, from about 20% to about 80%, from about 20% to about 85%, from about 20% to about 90%, from about 20% to about 25%, from about 20% to about 30%, from about 20% to about 35%, from about 20% to about 40%, from about 20% to about 45%, from about 20% to about 50%, from about 20% to about 55%, from about 20% to about 60%, from about 20% to about 65%, from about 20% to about 70%, from about 20% to about 75%, from about 20% to about 80%, from about 20% to about 85%, from about 20% to about 90%, from about 30% to about 35%, from about 30% to about 40%, from about 30% to about 45%, from about 30% to about 50%, from about 30% to about 55%, from about 30% to about 60%, from about 30% to about 65%, from about 30% to about 70%, from about 30% to about 75%, from about 30% to about 80%, from about 30% to about 85%, from about 30% to about 90%, from about 40% to about 45%, from about 40% to about 50%, from about 40% to about 55%, from about 40% to about 60%, from about 40% to about 65%, from about 40% to about 70%, from about 40% to about 75%, from about 40% to about 80%, from about 40% to about 85%, from about 40% to about 90%, from about 50% to about 55%, from about 50% to about 60%, from about 50% to about 65%, from about 50% to about 70%, from about 50% to about 75%, from about 50% to about 80%, from about 50% to about 85%, from about 50% to about 90%, from about 60% to about 65%, from about 60% to about 70%, from about 60% to about 75%, from about 60% to about 80%, from about 60% to about 85%, from about 60% to about 90%, from from about 70% to about 75%, from about 70% to about 80%, from about 70% to about 85%, from about 70% to about 90%, from about 80% to about 85%, and from about 80% to about 90%).

Other compounds may also be added to the formulations to increase the viscosity of the carrier. Examples of viscosity enhancing agents include, but are not limited to: polysaccharides, such as hyaluronic acid and its salts, chondroitin sulfate and its salts, dextrans, various polymers of the cellulose family; vinyl polymers; and acrylic acid polymers.

In some embodiments, the Nrf2 activator and/or mitochondrial protective agent may be administered by the use of or in the form of hydrogels, drug-eluting contact lenses, and nanosystems (liposomal systems, dendrimers, solid biodegradable nanoparticles, nanogels), and/or irrigating solutions.

### Packaging

The formulations of the present invention may be packaged as either a single dose product or a multi-dose product. The single dose product is sterile prior to opening of the package and all of the composition in the package is intended to be consumed in a single application to one or both eyes of a patient. The use of an antimicrobial preservative to maintain the sterility of the composition after the package is opened is generally unnecessary.

Multi-dose products are also sterile prior to opening of the package. However, because the container for the composition may be opened many times before all of the composition in the container is consumed, the multi-dose products must have sufficient antimicrobial activity to ensure that the compositions will not become contaminated by microbes as a result of the repeated opening and handling of the container. The level of antimicrobial activity required for this purpose is well known to those skilled in the art, and is specified in official publications, such as the United States Pharmacopoeia (“USP”) and corresponding publications in other countries. Detailed descriptions of the specifications for preservation of ophthalmic pharmaceutical products against microbial contamination and the procedures for evaluating the preservative efficacy of specific formulations are provided in those publications. In the United States, preservative efficacy standards are generally referred to as the “USP PET” requirements. (The acronym “PET” stands for “preservative efficacy testing.”)

The use of a single dose packaging arrangement eliminates the need for an antimicrobial preservative in the compositions, which is a significant advantage from a medical perspective, because conventional antimicrobial agents utilized to preserve ophthalmic compositions (e.g., benzalkonium chloride) may cause ocular irritation, particularly in patients suffering from dry eye conditions or pre-existing ocular irritation. However, the single dose packaging arrangements currently available, such as small volume plastic vials prepared by means of a process known as “form, fill and seal”, have several disadvantages for manufacturers and consumers. The principal disadvantages of the single dose packaging systems are the much larger quantities of packaging materials required, which is both wasteful and costly, and the inconvenience for the consumer. Also, there is a risk that consumers will not discard the single dose containers following application of one or two drops to the eyes, as they are instructed to do, but instead will save the opened container and any composition remaining therein for later use. This improper use of single dose products creates a risk of microbial contamination of the single dose product and an associated risk of ocular infection if a contaminated composition is applied to the eyes.

While the formulations of this invention are preferably formulated as “ready for use” aqueous solutions, alternative formulations are contemplated within the scope of this invention. Thus, for example, the active ingredients, surfactants, salts, chelating agents, or other components of the ophthalmic solution, or mixtures thereof, can be lyophilized or otherwise provided as a dried powder or tablet ready for dissolution (e.g., in deionized, or distilled) water. Because of the self-preserving nature of the solution, sterile water is not required.

### Kits

In still another embodiment, this invention provides kits for the packaging and/or storage and/or use of the formulations described herein, as well as kits for the practice of the methods described herein. Thus, for example, kits may comprise one or more containers containing one or more ophthalmic solutions, tablets, or capsules of this invention. The kits can be designed to facilitate one or more aspects of shipping, use, and storage.

The kits may optionally include instructional materials containing directions (i.e., protocols) disclosing means of use of the formulations provided therein. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g. CD ROM), and the like. Such media may include addresses to internet sites that provide such instructional materials.

### Methods of Use

The present invention relates to topical pharmaceutical compositions (e.g., oral, systemic or topical ophthalmic formulations) for useful to treat diseased or damaged corneal endothelium. In some embodiments, methods are provided for the treatment of the corneal endothelium in a subject in need of such treatment by administering the pharmaceutical compositions of the present invention orally or parenterally to a subject in need thereof. In some embodiments, methods are provided for the treatment of the corneal endothelium in a subject in need of such treatment by administering the ophthalmic formulations of the present invention directly to the eye of the subject. Diseases and conditions of the corneal endothelium include, but are not limited to, posterior polymorphous dystrophy, congenital hereditary endothelial dystrophy (CHED), iridocorneal endothelial (ICE) syndrome, Fuchs endothelial corneal Dystrophy (FECD), and pseudophakic bullous keratopathy (PBK).

The Nrf2 activator formulations of the invention are useful for the treatment the corneal endothelium. The Nrf2 activator formulations may further contain one or more (e.g., 1, 2, 3, 4, 5, 6, 7, 8, etc.) mitochondrial protective agents. According to some embodiments, the pharmaceutical compositions are formulated for oral or parenteral administration. According to some embodiments, the pharmaceutical compositions are formulated for topical administration to the eye (e.g., eye drops). According to some embodiments, the pharmaceutical compositions may further comprise a tear substitute.

According to some embodiments, methods are provided for treating the corneal endothelium in a subject in need thereof comprising administering to the subject a pharmaceutical composition comprising an effective amount of at least one (e.g., 1, 2, 3, 4, 5, 6, 7, 8, etc.) Nrf2 activator. In certain embodiments, the administration of Nrf2 activators to the subject in need of treatment of the corneal endothelium is also effective to mitigate or reduce one or more symptoms associated with a disease or condition of the corneal endothelium. The subject is preferably a human, but may be another mammal, for example a dog, a cat, a rabbit, a mouse, a rat, or a non-human primate.

According to some embodiments, methods are provided for treating the corneal endothelium in a subject in need thereof comprising administering to the eye surface of the subject a pharmaceutical composition comprising an effective amount of at least one (e.g., 1, 2, 3, 4, 5, 6, 7, 8, etc.) Nrf2 activator. In certain embodiments, the administration of Nrf2 activators to the eye of a subject in need of treatment of the corneal endothelium is also effective to mitigate or reduce one or more symptoms associated with a disease or condition of the corneal endothelium. The subject is preferably a human, but may be another mammal, for example a dog, a cat, a rabbit, a mouse, a rat, or a non-human primate.

The formulations of the present invention contain an amount of one or more Nrf2 activators, and optionally one or more additional active ingredients, effective for the intended use. Particular dosages are also selected based on a number of factors including the age, sex, species and condition of the subject. Effective amounts can also be extrapolated from dose-response curves derived from in vitro test systems or from animal models. The term “effective amount” means an amount of Nrf2 activators that is sufficient to treat or eliminate or reduce a symptom as a result of disease or damage to the corneal endothelium. In certain embodiments, the effective amount is the amount sufficient for the treatment of a disease, a condition, or damage to the corneal endothelium. “Treatment” in this context refers to reducing or ameliorating at least one symptom as a result of disease or damage to the corneal endothelium. “Prevention” in this context refers to a reduction in the frequency of, or a delay in the onset of, symptoms associated with a disease or condition, relative to a subject who does not receive the composition. The invention features methods of treating the corneal endothelium in a subject comprising use of the novel formulations described above. For example, a method of treating the corneal endothelium may comprise administering to the eye surface of the subject a pharmaceutical composition comprising an effective amount of at least one Nrf2 activator and a tear substitute in a pharmaceutically acceptable carrier.

The mitochondrially targeted antioxidant formulations of the invention are useful for the treatment of the corneal endothelium. The mitochondrially targeted antioxidants formulations may further contain one or more (e.g., 1, 2, 3, 4, 5, 6, 7, 8, etc.) Nrf2 activators. According to some embodiments, the pharmaceutical compositions are formulated for oral or parenteral administration. According to some embodiments, the pharmaceutical compositions are formulated for topical administration to the eye (e.g., eye drops). According to some embodiments, the pharmaceutical compositions may further comprise a tear substitute.

According to some embodiments, methods are provided for treating the corneal endothelium in a subject in need thereof comprising administering to the subject a pharmaceutical composition comprising an effective amount of at least one (e.g., 1, 2, 3, 4, 5, 6, 7, 8, etc.) mitochondrially targeted antioxidant. In certain embodiments, the administration of mitochondrially targeted antioxidants to the subject in need of treatment of the corneal endothelium is also effective to mitigate or reduce one or more symptoms associated with disease or damage to the corneal endothelium. The subject is preferably a human, but may be another mammal, for example a dog, a cat, a rabbit, a mouse, a rat, or a non-human primate.

Methods are provided for treating the corneal endothelium in a subject in need thereof are carried out by administering to the eye surface of the subject a pharmaceutical composition comprising an effective amount of at least one (e.g., 1, 2, 3, 4, 5, 6, 7, 8, etc.) mitochondrially targeted antioxidant. The administration of mitochondrially targeted antioxidants to the eye of a subject in need of treatment of the corneal endothelium is also effective to mitigate or reduce one or more symptoms associated with disease or damage to the corneal endothelium. The subject is preferably a human, but may be another mammal, for example a dog, a cat, a rabbit, a mouse, a rat, or a non-human primate.

Methods are provided for treating or preventing pterygia in a subject in need thereof comprising administering to the subject a pharmaceutical composition comprising an effective amount of at least one Nrf2 activator and/or mitochondrial protective agent. In certain embodiments, the administration of Nrf2 activator and/or mitochondrial protective agent (e.g., mitochondrially targeted antioxidants) to the subject in need of treatment or prevention of pterygia is also effective to mitigate or reduce one or more symptoms associated with pterygia. The subject is preferably a human, but may be another mammal, for example a dog, a cat, a rabbit, a mouse, a rat, or a non-human primate.

Methods are provided for treating or preventing pterygia in a subject in need thereof comprising administering to the eye surface of the subject a pharmaceutical composition comprising an effective amount of at least one Nrf2 activator and/or mitochondrial protective agent. In certain embodiments, the administration of Nrf2 activator and/or mitochondrial protective agent (e.g., mitochondrially targeted antioxidants) to the eye of a subject in need of treatment or prevention of pterygia is also effective to mitigate or reduce one or more symptoms associated with pterygia. The subject is preferably a human, but may be another mammal, for example a dog, a cat, a rabbit, a mouse, a rat, or a non-human primate.

Methods for treating or preventing pseudophakic bullous keratopathy (PBK) or aphakic bullous keratopathy (ABK) in a subject in need thereof are carried out by administering to the subject a pharmaceutical composition comprising an effective amount of at least one Nrf2 activator and/or mitochondrial protective agent. In certain embodiments, the administration of Nrf2 activator and/or mitochondrial protective agent (e.g., mitochondrially targeted antioxidants) to the subject in need of treatment or prevention of pseudophakic bullous keratopathy (PBK) or aphakic bullous keratopathy (ABK) is also effective to mitigate or reduce one or more symptoms associated with pseudophakic bullous keratopathy (PBK) or aphakic bullous keratopathy (ABK). The subject is preferably a human, but may be another mammal, for example a dog, a cat, a rabbit, a mouse, a rat, or a non-human primate.

Methods for treating or preventing pseudophakic bullous keratopathy (PBK) or aphakic bullous keratopathy (ABK) in a subject in need thereof involve administering to the eye surface of the subject a pharmaceutical composition comprising an effective amount of at least one Nrf2 activator and/or mitochondrial protective agent. In certain embodiments, the administration of Nrf2 activator and/or mitochondrial protective agent (e.g., mitochondrially targeted antioxidants) to the eye of a subject in need of treatment or prevention of pseudophakic bullous keratopathy (PBK) or aphakic bullous keratopathy (ABK) is also effective to mitigate or reduce one or more symptoms associated with pseudophakic bullous keratopathy (PBK) or aphakic bullous keratopathy (ABK). The subject is preferably a human, but may be another mammal, for example a dog, a cat, a rabbit, a mouse, a rat, or a non-human primate.

According to some embodiments, methods are provided for treating or preventing endothelial cell loss caused by intraocular surgery in a subject in need thereof comprising administering to the subject a pharmaceutical composition comprising an effective amount of at least one Nrf2 activator and/or mitochondrial protective agent. In certain embodiments, methods are provided for the prophylactic treatment of endothelial cell loss before, during, and after cataract surgery comprising administering to the subject a pharmaceutical composition comprising an effective amount of at least one Nrf2 activator and/or mitochondrial protective agent. The subject is preferably a human, but may be another mammal, for example a dog, a cat, a rabbit, a mouse, a rat, or a non-human primate.

According to some embodiments, methods are provided for treating or preventing endothelial cell loss caused by intraocular surgery in a subject in need thereof comprising administering to the eye surface of the subject a pharmaceutical composition comprising an effective amount of at least one Nrf2 activator and/or mitochondrial protective agent. In certain embodiments, methods are provided for the prophylactic treatment of endothelial cell loss before, during, and after cataract surgery comprising administering to the eye surface of the subject a pharmaceutical composition comprising an effective amount of at least one Nrf2 activator and/or mitochondrial protective agent. The at least one Nrf2 activator and/or mitochondrial protective agent may be added to the irrigation solutions routinely used during phacoemulcification and/or vitreoretinal procedures. The subject is preferably a human, but may be another mammal, for example a dog, a cat, a rabbit, a mouse, a rat, or a non-human primate.

In some embodiments, the at least one Nrf2 activator and/or mitochondrial protective agent is administered alone or in combination with antioxidant peptides. The preferred antioxidant peptides are Szeto-Schiller (SS) peptides and their functional analogs. These compounds have alternating aromatic residues and basic amino acids. In particular, peptides having tyrosine (Tyr) or dimethyltyrosine (Dmt) analogs can scavenge oxyradicals. These compounds inhibit oxidation of low-density lipoproteins. SS-peptides include compounds such as D-Arg-Dmt-Lys-Phe-NH2) and Dmt-D-Arg-Phe-Lys-NH2. In addition to the Tyr and Dmt containing SS-peptides, tryptophan containing SS-peptides are also useful in the current invention. Finally, the amino acids found in the SS-peptides may be L or D and may be naturally occurring, non-naturally occurring and derivatives of naturally occurring amino acids. In particular, the SS-peptides disclosed in PCT published application WO 2005/072295 are suitable for use in the current invention. The entire disclosure of WO 2005/072295, published on Aug. 11, 2005 is incorporated herein by reference.

According to some embodiments, methods are provided for treating or preventing endothelial cell loss caused by intraocular surgery in a subject in need thereof comprising administering to the eye surface of the subject a pharmaceutical composition comprising an effective amount of at least one Szeto-Schiller (SS) peptides. In certain embodiments, methods are provided for the prophylactic treatment of endothelial cell loss before, during, and after cataract surgery comprising administering to the eye surface of the subject a pharmaceutical composition comprising an effective amount of at least one Szeto-Schiller (SS) peptides. The at least one Szeto-Schiller (SS) peptides may be added to the irrigation solutions routinely used during phacoemulsification and/or vitreoretinal procedures. The subject is preferably a human, but may be another mammal, for example a dog, a cat, a rabbit, a mouse, a rat, or a non-human primate.

In some cases, the Nrf2 activators described herein are combined with corneal storage media such as Optisol to enhance endothelial cell survival prior to and during transplantation.

The formulations of the present invention contain an amount of mitochondrially targeted antioxidants, and optionally one or more additional active ingredients (e.g., Nrf2 activators), that is effective for the intended use. Particular dosages are also selected based on a number of factors including the age, sex, species and condition of the subject. Effective amounts can also be extrapolated from dose-response curves derived from in vitro test systems or from animal models. The term “effective amount” means an amount of mitochondrially targeted antioxidants that is sufficient to treat or eliminate or reduce a symptom as a result of disease or damage to the corneal endothelium. In certain embodiments, the effective amount is the amount sufficient for the treatment of the corneal endothelium. “Treatment” in this context refers to reducing or ameliorating at least one symptom as a result of a disease, a condition, or damage to the corneal endothelium. “Prevention” in this context refers to a reduction in the frequency of, or a delay in the onset of, symptoms associated a disease or condition of the corneal endothelium, relative to a subject who does not receive the composition.

The effective amount of the active agents in the formulation will depend on absorption, inactivation, and excretion rates of the drug as well as the delivery rate of the compound from the formulation. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

The dosage of any compound of the present invention will vary depending on the symptoms, age and other physical characteristics of the patient, the nature and severity of the disorder to be treated or prevented, the degree of comfort desired, the route of administration, and the form of the supplement. Any of the subject formulations may be administered in a single dose or in divided doses. Dosages for the formulations of the present invention may be readily determined by techniques known to those of skill in the art or as taught herein.

An effective dose or amount, and any possible effects on the timing of administration of the formulation, may need to be identified for any particular formulation of the present invention. This may be accomplished by routine experiment as described herein. The effectiveness of any formulation and method of treatment or prevention may be assessed by administering the formulation and assessing the effect of the administration by measuring one or more indices associated with the efficacy of the active agent and with the degree of comfort to the patient, as described herein, and comparing the post-treatment values of these indices to the values of the same indices prior to treatment or by comparing the post-treatment values of these indices to the values of the same indices using a different formulation.

The precise time of administration and amount of any particular formulation that will yield the most effective treatment in a given patient will depend upon the activity, pharmacokinetics, and bioavailability of a particular compound, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

The combined use of several active agents formulated into the compositions of the present invention may reduce the required dosage for any individual component because the onset and duration of effect of the different components may be complimentary. In such combined therapy, the different active agents may be delivered together or separately, and simultaneously or at different times within the day.

### Fuchs Endothelial Corneal Dystrophy (FECD)

The invention relates to activating binding of nuclear factor E2-related factor 2 (Nrf2) to the antioxidant response element (ARE) in methods for treating Fuchs endothelial corneal dystrophy (FECD). Compositions comprising Nrf2 activators are used in the treatment of FECD. The data presented herein demonstrates that there is downregulation of Nrf2-ARE-driven antioxidant and oxidative stress-related gene expression, a decline in the levels of the major transcription factor known to regulate Nrf2-ARE-dependent antioxidants, and an increase in oxidative mtDNA damage in FECD. The suboptimal Nrf2-regulated antioxidant defense contributes to the oxidant-antioxidant imbalance seen in FECD. Oxidative stress was found to be directly associated with FECD pathogenesis and represents a target for therapy to prevent or inhibit this common ocular condition. Prevention of or reduction of corneal endothelial cell loss in early as well as late stages of the disease by reversing oxidant-antioxidant imbalance according to the invention delays or bypasses completely the need for corneal transplantation.

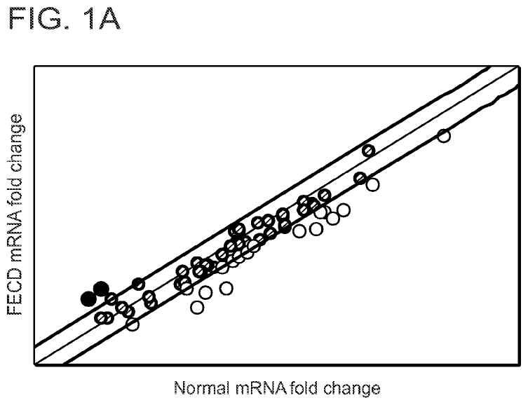

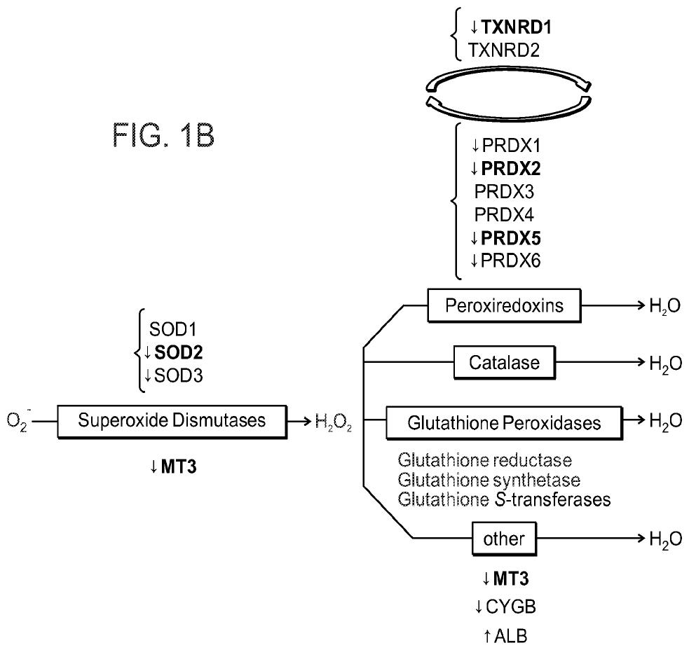

FECD pathogenesis was determined to be associated with the deficiency of the major transcription factor, called NF-E2 related factor-2 (Nrf2), in FECD. Nrf2 controls the inducible expression of numerous antioxidants and phase 2 detoxification genes, such as glutathione S-transferase, heme oxygenase-1, and NAD(P)H:quinone oxidoreductase 1 (NQO1). Nrf2 is responsible for a coordinated upregulation of defense enzymes that protect cells from excess of reactive oxygen species and apoptosis (See FIGS. 1-3). Nrf2 and the antioxidant defense regulated by Nrf2 are deficient in FECD corneal endothelium. The findings of a dysregulated Nrf2-controlled pathway are of critical relevance in designing the pharmacologic targets to diminish the loss endothelial cells in FECD. The Nrf2 pathway has not been described in FECD or corneal endothelium and targeting it presents a novel therapeutic strategy. The methods of the invention are advantageous over other approaches because targeting Nrf2 leads to upregulation of a wide range of antioxidants. In contrast, single antioxidant molecules (a vitamin, for example) may not be efficacious or potent enough to ameliorate the deficiencies seen in FECD endothelium.

The Nrf2 activator formulations of the invention are useful for the treatment, reduction, or prevention of the symptoms of FECD. The Nrf2 activator formulations optionally contain one or more (e.g., 1, 2, 3, 4, 5, 6, 7, 8, etc.) mitochondrial protective agents. According to some embodiments, the pharmaceutical compositions are formulated for oral or parenteral administration. According to some embodiments, the pharmaceutical compositions are formulated for topical administration to the eye (e.g., eye drops). According to some embodiments, the pharmaceutical compositions may further comprise a tear substitute.

According to some embodiments, methods are provided for treating or preventing FECD in a subject in need thereof comprising administering to the eye surface of the subject a pharmaceutical composition comprising an effective amount of at least one (e.g., 1, 2, 3, 4, 5, 6, 7, 8, etc.) Nrf2 activator. In certain embodiments, the administration of Nrf2 activators to the eye of a subject in need of treatment or prevention of FECD is also effective to mitigate or reduce one or more symptoms associated with FECD.

The formulations of the present invention contain an amount of Nrf2 activators, and optionally one or more additional active ingredients, effective for the intended use. Particular dosages are also selected based on a number of factors including the age, sex, species and condition of the subject. Effective amounts can also be extrapolated from dose-response curves derived from in vitro test systems or from animal models. The term “effective amount” means an amount of Nrf2 activators that is sufficient to treat or eliminate or reduce a symptom of FECD. In certain embodiments, the effective amount is the amount sufficient for the treatment or prevention of FECD. “Treatment” in this context refers to reducing or ameliorating at least one symptom of FECD. “Prevention” in this context refers to a reduction in the frequency of, or a delay in the onset of, symptoms associated with FECD, relative to a subject who does not receive the composition. The invention features methods of treating or preventing FECD in a subject comprising use of the novel formulations described above. For example, a method of treating FECD may comprise administering to the eye surface of the subject a pharmaceutical composition comprising an effective amount of at least one Nrf2 activator and a tear substitute in a pharmaceutically acceptable carrier.

The mitochondrially targeted antioxidant formulations of the invention are useful for the treatment and prevention of the symptoms of FECD. The mitochondrially targeted antioxidants formulations may further contain one or more (e.g., 1, 2, 3, 4, 5, 6, 7, 8, etc.) Nrf2 activators. According to some embodiments, the pharmaceutical compositions are formulated for oral or parenteral administration. According to some embodiments, the pharmaceutical compositions are formulated for topical administration to the eye (e.g., eye drops). According to some embodiments, the pharmaceutical compositions may further comprise a tear substitute.

According to some embodiments, methods are provided for treating or preventing FECD in a subject in need thereof comprising administering to the eye surface of the subject a pharmaceutical composition comprising an effective amount of at least one (e.g., 1, 2, 3, 4, 5, 6, 7, 8, etc.) mitochondrially targeted antioxidant. In certain embodiments, the administration of mitochondrially targeted antioxidants to the eye of a subject in need of treatment or prevention of FECD is also effective to mitigate or reduce one or more symptoms associated with FECD. The subject is preferably a human, but may be another mammal, for example a dog, a cat, a rabbit, a mouse, a rat, or a non-human primate.

The formulations of the present invention contain an amount of mitochondrially targeted antioxidants, and optionally one or more additional active ingredients (e.g., Nrf2 activators), that is effective for the intended use. Particular dosages are also selected based on a number of factors including the age, sex, species and condition of the subject. Effective amounts can also be extrapolated from dose-response curves derived from in vitro test systems or from animal models. The term “effective amount” means an amount of mitochondrially targeted antioxidants that is sufficient to treat or eliminate or reduce a symptom of FECD. In certain embodiments, the effective amount is the amount sufficient for the treatment or prevention of FECD. “Treatment” in this context refers to reducing or ameliorating at least one symptom of FECD. “Prevention” in this context refers to a reduction in the frequency of, or a delay in the onset of, symptoms associated with FECD, relative to a subject who does not receive the composition.

### Pterygia

Methods are provided for treating or preventing pterygia in a subject in need thereof comprising administering to the eye surface of the subject a pharmaceutical composition comprising an effective amount of at least one Nrf2 activator and/or mitochondrial protective agent. In certain embodiments, the administration of Nrf2 activator and/or mitochondrial protective agent (e.g., mitochondrially targeted antioxidants) to the eye of a subject in need of treatment or prevention of pterygia is also effective to mitigate or reduce one or more symptoms associated with pterygia. The subject is preferably a human, but may be another mammal, for example a dog, a cat, a rabbit, a mouse, a rat, or a non-human primate.

### Pseudophakic Bullous Keratopathy (PBK) or Aphakic Bullous Keratopathy (ABK)

Methods are provided for treating or preventing pseudophakic bullous keratopathy (PBK) or aphakic bullous keratopathy (ABK) in a subject in need thereof comprising administering to the eye surface of the subject a pharmaceutical composition comprising an effective amount of at least one Nrf2 activator and/or mitochondrial protective agent. In certain embodiments, the administration of Nrf2 activator and/or mitochondrial protective agent (e.g., mitochondrially targeted antioxidants) to the eye of a subject in need of treatment or prevention of pseudophakic bullous keratopathy (PBK) or aphakic bullous keratopathy (ABK) is also effective to mitigate or reduce one or more symptoms associated with pseudophakic bullous keratopathy (PBK) or aphakic bullous keratopathy (ABK). The subject is preferably a human, but may be another mammal, for example a dog, a cat, a rabbit, a mouse, a rat, or a non-human primate.

The Nrf2 activator formulations of the invention are useful for the treatment and prevention of the symptoms of PBK. The Nrf2 activator formulations may further contain one or more (e.g., 1, 2, 3, 4, 5, 6, 7, 8, etc.) mitochondrial protective agents. According to some embodiments, the pharmaceutical compositions are formulated for oral or parenteral administration. According to some embodiments, the pharmaceutical compositions are formulated for topical administration to the eye (e.g., eye drops). According to some embodiments, the pharmaceutical compositions may further comprise a tear substitute.

According to some embodiments, methods are provided for treating or preventing PBK in a subject in need thereof comprising administering to the eye surface of the subject a pharmaceutical composition comprising an effective amount of at least one (e.g., 1, 2, 3, 4, 5, 6, 7, 8, etc.) Nrf2 activator. In certain embodiments, the administration of Nrf2 activators to the eye of a subject in need of treatment or prevention of PBK is also effective to mitigate or reduce one or more symptoms associated with PBK. The subject is preferably a human, but may be another mammal, for example a dog, a cat, a rabbit, a mouse, a rat, or a non-human primate.

The formulations of the present invention contain an amount of Nrf2 activators, and optionally one or more additional active ingredients, that are effective for the intended use. Particular dosages are also selected based on a number of factors including the age, sex, species and condition of the subject. Effective amounts can also be extrapolated from dose-response curves derived from in vitro test systems or from animal models. The term “effective amount” means an amount of Nrf2 activators that is sufficient to treat or eliminate or reduce a symptom of PBK. In certain embodiments, the effective amount is the amount sufficient for the treatment or prevention of PBK. “Treatment” in this context refers to reducing or ameliorating at least one symptom of PBK. “Prevention” in this context refers to a reduction in the frequency of, or a delay in the onset of, symptoms associated with PBK, relative to a subject who does not receive the composition. The invention features methods of treating or preventing PBK in a subject comprising use of the novel formulations described above. For example, a method of treating PBK may comprise administering to the eye surface of the subject a pharmaceutical composition comprising an effective amount of at least one Nrf2 activator and a tear substitute in a pharmaceutically acceptable carrier.

The mitochondrially targeted antioxidant formulations of the invention are useful for the treatment and prevention of the symptoms of PBK. The mitochondrially targeted antioxidants formulations may further contain one or more (e.g., 1, 2, 3, 4, 5, 6, 7, 8, etc.) Nrf2 activators. According to some embodiments, the pharmaceutical compositions are formulated for oral or parenteral administration. According to some embodiments, the pharmaceutical compositions are formulated for topical administration to the eye (e.g., eye drops). According to some embodiments, the pharmaceutical compositions may further comprise a tear substitute.

According to some embodiments, methods are provided for treating or preventing PBK in a subject in need thereof comprising administering to the eye surface of the subject a pharmaceutical composition comprising an effective amount of at least one (e.g., 1, 2, 3, 4, 5, 6, 7, 8, etc.) mitochondrially targeted antioxidant. In certain embodiments, the administration of mitochondrially targeted antioxidants to the eye of a subject in need of treatment or prevention of PBK is also effective to mitigate or reduce one or more symptoms associated with PBK. The subject is preferably a human, but may be another mammal, for example a dog, a cat, a rabbit, a mouse, a rat, or a non-human primate.

The formulations of the present invention contain an amount of mitochondrially targeted antioxidants, and optionally one or more additional active ingredients (e.g., Nrf2 activators), that is effective for the intended use. Particular dosages are also selected based on a number of factors including the age, sex, species and condition of the subject. Effective amounts can also be extrapolated from dose-response curves derived from in vitro test systems or from animal models. The term “effective amount” means an amount of mitochondrially targeted antioxidants that is sufficient to treat or eliminate or reduce a symptom of PBK. In certain embodiments, the effective amount is the amount sufficient for the treatment or prevention of PBK. “Treatment” in this context refers to reducing or ameliorating at least one symptom of PBK. “Prevention” in this context refers to a reduction in the frequency of, or a delay in the onset of, symptoms associated with PBK, relative to a subject who does not receive the composition.

### Endothelial Cell Loss

According to some embodiments, methods are provided for treating or preventing endothelial cell loss caused by intraocular surgery in a subject in need thereof comprising administering to the eye surface of the subject a pharmaceutical composition comprising an effective amount of at least one Nrf2 activator and/or mitochondrial protective agent. In certain embodiments, methods are provided for the prophylactic treatment of endothelial cell loss before, during, and after cataract surgery comprising administering to the eye surface of the subject a pharmaceutical composition comprising an effective amount of at least one Nrf2 activator and/or mitochondrial protective agent. The at least one Nrf2 activator and/or mitochondrial protective agent may be added to the irrigation solutions routinely used during phacoemulcification, vitreoretinal, and/or intraocular procedures. The subject is preferably a human, but may be another mammal, for example a dog, a cat, a rabbit, a mouse, a rat, or a non-human primate.

According to some embodiments, the compositions and formulations of the present invention are suitable for use as intraocular solutions (e.g., irrigation solutions) used during surgeries associated with the loss or risk of loss of endothelial cells (e.g., intraocular surgery). The compositions and formulations of the present invention may be used in methods to arrest or prevent endothelial cell loss associated with such surgeries.

### Antioxidant Peptides

At least one Nrf2 activator and/or mitochondrial protective agent is administered alone or in combination with antioxidant peptides. The preferred antioxidant peptides are Szeto-Schiller (SS) peptides and their functional analogs. These compounds have alternating aromatic residues and basic amino acids. In particular, peptides having tyrosine (Tyr) or dimethyltyrosine (Dmt) analogs can scavenge oxyradicals. These compounds inhibit oxidation of low-density lipoproteins. SS-peptides include compounds such as D-Arg-Dmt-Lys-Phe-NH2) and Dmt-D-Arg-Phe-Lys-NH2. In addition to the Tyr and Dmt containing SS-peptides, tryptophan containing SS-peptides are also useful in the current invention. Finally, the amino acids found in the SS-peptides may be L or D and may be naturally occurring, non-naturally occurring and derivatives of naturally occurring amino acids. In particular, the SS-peptides disclosed in PCT published application WO 2005/072295 are suitable for use in the current invention. The entire disclosure of WO 2005/072295, published on Aug. 11, 2005 is incorporated herein by reference.

According to some embodiments, methods are provided for treating or preventing endothelial cell loss caused by intraocular surgery in a subject in need thereof comprising administering to the eye surface of the subject a pharmaceutical composition comprising an effective amount of at least one Szeto-Schiller (SS) peptides. In certain embodiments, methods are provided for the prophylactic treatment of endothelial cell loss before, during, and after cataract surgery comprising administering to the eye surface of the subject a pharmaceutical composition comprising an effective amount of at least one Szeto-Schiller (SS) peptides. The at least one Szeto-Schiller (SS) peptides may be added to the irrigation solutions routinely used during phacoemulsification and/or vitreoretinal procedures. The subject is preferably a human, but may be another mammal, for example a dog, a cat, a rabbit, a mouse, a rat, or a non-human primate.

### Therapeutic Administration

The effective amount of the active agents in the formulation will depend on absorption, inactivation, and excretion rates of the drug as well as the delivery rate of the compound from the formulation. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

The dosage of any compound of the present invention will vary depending on the symptoms, age and other physical characteristics of the patient, the nature and severity of the disorder to be treated or prevented, the degree of comfort desired, the route of administration, and the form of the supplement. Any of the subject formulations may be administered in a single dose or in divided doses. Dosages for the formulations of the present invention may be readily determined by techniques known to those of skill in the art or as taught herein.

An effective dose or amount, and any possible effects on the timing of administration of the formulation, may need to be identified for any particular formulation of the present invention. This may be accomplished by routine experiment as described herein. The effectiveness of any formulation and method of treatment or prevention may be assessed by administering the formulation and assessing the effect of the administration by measuring one or more indices associated with the efficacy of the active agent and with the degree of comfort to the patient, as described herein, and comparing the post-treatment values of these indices to the values of the same indices prior to treatment or by comparing the post-treatment values of these indices to the values of the same indices using a different formulation.

The precise time of administration and amount of any particular formulation that will yield the most effective treatment in a given patient will depend upon the activity, pharmacokinetics, and bioavailability of a particular compound, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

The combined use of several active agents formulated into the compositions of the present invention may reduce the required dosage for any individual component because the onset and duration of effect of the different components may be complimentary. In such combined therapy, the different active agents may be delivered together or separately, and simultaneously or at different times within the day.

### Definitions

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only not intended to be limiting. Other features and advantages of the invention will be apparent from the following detailed description and claims.

For the purposes of promoting an understanding of the embodiments described herein, reference will be made to preferred embodiments and specific language will be used to describe the same. The terminology used herein is for the purpose of describing particular embodiments only, and is not intended to limit the scope of the present invention. As used throughout this disclosure, the singular forms “a,” “an,” and “the” include plural reference unless the context clearly dictates otherwise. Thus, for example, a reference to “a composition” includes a plurality of such compositions, as well as a single composition, and a reference to “a therapeutic agent” is a reference to one or more therapeutic and/or pharmaceutical agents and equivalents thereof known to those skilled in the art, and so forth.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “aqueous” typically denotes an aqueous composition wherein the carrier is to an extent of >50%, more preferably >75% and in particular >90% by weight water.

The phrase “effective amount” is an art-recognized term, and refers to an amount of an agent that, when incorporated into a pharmaceutical composition of the present invention, produces some desired effect at a reasonable benefit/risk ratio applicable to any medical treatment. In certain embodiments, the term refers to that amount necessary or sufficient to eliminate, reduce or maintain (e.g., prevent the spread of) a symptom of a disease or condition of the corneal endothelium (e.g., FECD). One of skill in the art may empirically determine the effective amount of a particular agent without necessitating undue experimentation.

A “patient,” “subject,” or “host” to be treated by the subject method refers to either a human or non-human animal, such as primates, mammals, and vertebrates.

The phrase “pharmaceutically acceptable” is art-recognized and refers to compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” is art-recognized, and refers to, for example, pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body. Each carrier must be “acceptable” in the sense of being compatible with the other ingredients of the supplement and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “preventing,” when used in relation to a condition, such as ocular allergy is art-recognized, and refers to administration of a composition which reduces the frequency of, or delays the onset of, symptoms of a medical condition in a subject relative to a subject which does not receive the composition.

As used herein, the term “tear substitute” refers to molecules or compositions which lubricate, “wet,” approximate the consistency of endogenous tears, aid in natural tear build-up, or otherwise provide temporary relief of dry eye symptoms and conditions upon ocular administration.

The term “treating” is an art-recognized term which refers to curing as well as ameliorating at least one symptom of any condition or disease.

Abbreviations used: 8-OHdG, 8-hydroxy-2′-deoxyguanosine; ALB, albumin; ARE, antioxidant response element; B2M, β2-microglobulin; CE, corneal endothelial (endothelium); dUTP, deoxyuridine-5′-triphosphate-digoxigenin; FECD, Fuchs endothelial corneal dystrophy; H₂O₂, hydrogen peroxide; HCECi, immortalized human corneal endothelial cells; MT3, metallothionein 3; NCF2, neutrophil cytosolic factor 2; Nrf2, nuclear factor erythroid 2-related factor 2; PBK, pseudophakic bullous keratopathy; PRDXs, peroxiredoxins; ROS, reactive oxygen species; RPL13a, ribosomal protein L13a; SOD, superoxide dismutase; TdT, terminal deoxyribonucleotidyl transferase; TUNEL, terminal deoxynucleotidyl transferase Biotin dUTP nick end labeling; TXNRD1, thioredoxin reductase 1; ZO-1, zonula occludens 1.

All publications and patents mentioned herein are hereby incorporated by reference in their entirety as if each individual publication or patent was specifically and individually indicated to be incorporated by reference. In case of conflict, the present application, including any definitions herein, will control.

### EXAMPLES

It is understood that modifications which do not substantially affect the activity of the various embodiments of this invention are also provided within the definition of the invention provided herein. Accordingly, the disclosed examples are intended to illustrate but not limit the present invention. While the claimed invention has been described in detail and with reference to specific embodiments thereof, it will be apparent to one of ordinary skill in the art that various changes and modifications can be made to the claimed invention without departing from the spirit and scope thereof. Thus, for example, those skilled in the art will recognize, or be able to ascertain, using no more than routine experimentation, numerous equivalents to the specific substances and procedures described herein. Such equivalents are considered to be within the scope of this invention, and are covered by the following claims.

### Example 1: Evidence of Oxidative Stress in the Pathogenesis of Fuchs Endothelial Corneal Dystrophy

The data presented shows a decrease in the antioxidant response element (ARE)-driven antioxidants in FECD CE. The data also demonstrate that nuclear factor erythroid 2-related factor 2 (Nrf2), a transcription factor that binds ARE and activates antioxidant defense, is downregulated in FECD endothelium. Significantly higher levels of oxidative DNA damage and apoptosis were detected in FECD endothelium as compared to normal controls and to pseudophakic bullous keratopathy (PBK) (iatrogenic CE cell loss) specimens. A marker of oxidative DNA damage, 8-hydroxy-2′-deoxyguanosine (8-OHdG), colocalized to mitochondria, indicating that the mitochondrial genome is the specific target of oxidative stress in FECD. Oxidative DNA damage was not detected in PBK corneas, while it colocalized with TUNEL-positive cells in FECD samples. Ex vivo, oxidative stress caused characteristic morphological changes and apoptosis of CE, suggestive of findings that characterize FECD in vivo. These data indicate that suboptimal Nrf2-regulated defense accounts for oxidant-antioxidant imbalance in FECD, which in turn leads to oxidative DNA damage and apoptosis. The data provides evidence that oxidative stress plays a key role in FECD pathogenesis.

Studies were carried out to determine whether FECD is associated with oxidant-antioxidant imbalance, and if so, whether oxidant-induced DNA damage (specifically mitochondrial DNA damage) is present in diseased endothelium. Antioxidant gene profiling was performed, comparing FECD CE to normal, and detected a decreased antioxidant defense system in FECD. Oxidative stress was found to be the inciting factor of the pathophysiological processes seen in FECD: 1) There is an oxidant-antioxidant imbalance seen in FECD as compared to normal CE. 2) There is an accumulation of oxidized DNA lesions in FECD CE as compared to normal subjects. 3) Oxidative stress in vitro induces the characteristic morphological changes and apoptosis in CE seen in FECD. The correlation between oxidative damage and apoptosis in FECD CE provides key evidence in the role of oxidative stress in the pathogenesis of this age-related, chronic, corneal condition.

The following materials and methods were used to generate the data described herein.

Human Tissue. After surgical removal, one-third of the FECD and PBK corneal button was used for histopathological confirmation of the diagnosis and two-thirds was used for the study, and immediately stored in corneal storage medium (Optisol-GS; Bausch & Lomb) at 4° C. Fresh normal human corneal buttons from National Disease Research Interchange (NDRI) and Tissue Banks International (TBI) were used as controls. Table I presents information regarding the tissue samples used. Normal donors were gender and decade matched with FECD and PBK donors.

TABLE I
Donor Information

| | FECD* | Normal | PBK** |
|---|---|---|---|
| Average age^{†} | 67 ± 11 | 66 ± 6 | 67 ± 22 |
| Sex (F/M) | 17/4 | 14/7 | 2/2 |

*FECD: Fuchs endothelial corneal dystrophy
**PBK: pseudophakic bullous keratopathy
^{†}Average age in years with standard deviation shown

Human Corneal Endothelial Cell Culture. An immortalized adult human corneal endothelial cell line (HCECi), cells were grown in T25 culture flasks in cell growth medium containing 8% FBS. The culture medium of subconfluent cells was replaced with serum-free medium (OptiMEM-1; Invitrogen-Life Technologies) alone or supplemented with H₂O₂ (200 μM) and incubated for 2 hours at 37° C. At the end of the treatment, cell viability was evaluated by trypan blue staining.

PCR Arrays. Under a dissecting microscope Descemet's membrane, along with the CE cell layer, was dissected from the stroma of corneal buttons. Total RNA was extracted from normal and FECD samples using the RNeasy Micro kit (Qiagen). cDNA was prepared with the RT2 First Strand Kit (SABiosciences) and loaded on the Human Oxidative Stress and Antioxidant Defense RT2 Profiler PCR Array (SABiosciences). The PCR arrays were run on the ABI Prism 7900 HT (Applied Biosystems) sequence detection system. Dissociation curves showed specificity of the amplified product, except for one gene, the neutrophil cytosolic factor 1, which was excluded from the study. The average expression of the housekeeping genes β₂-microglobulin (B2M), ribosomal protein L13a (RPL13a), and β-actin was used for normalization. Data analysis was performed on a data analysis template provided by SABiosciences. The comparative Ct method was used to calculate the mRNA fold-change in FECD CE relative to normal.

Identification of Antioxidant Response Element (ARE) Consensus Sequences. The National Center for Biotechnology Information sequences of genes that were more than 2-fold up- or downregulated in FECD compared to normal, were retrieved. Using Human BLAT, a search for the consensus ARE core sequence (A/G TGACNNN GC) was carried out up to 5,000 bp upstream of the transcription start site. Complement sequences were also searched.

Western Blot Analysis. Western blot experiments were conducted using known methods. Blots were incubated overnight with rabbit polyclonal anti-Nrf2 (1:100; Santa Cruz Biotechnology), followed by incubation with HRP-conjugated secondary antibody. Mouse anti-(3-actin (1:6,000; Sigma Aldrich) was used to normalize protein loading. Proteins were detected with an enhanced chemiluminescence detection kit (SuperSignal). Densitometry was performed with Kodak Digital Science 1D software.

Quantitative analysis of 8-hydroxy-2′-deoxyguanosine (8-OHdG). DNA was extracted from normal and FECD CE, and from H₂O₂-treated and non-treated HCECi using QIAamp DNA Micro Kit (Qiagen) and eluted in distilled water. The DNA concentration of each sample was measured with the Nanodrop ND-1000 (Thermo Scientific). The DNA was digested with nuclease P1 (USBiological) then treated with alkaline phosphatase (Roche), and filtered through the Microcon YM-10 ultrafiltration membrane (Millipore). The content of 8-OHdG in each sample was determined using the highly sensitive 8-OHdG ELISA kit (Northwest Life Sciences Specialties LLC). To normalize between samples, the 8-OHdG content of each sample was divided by the amount of DNA loaded, in nanograms.

Real-time PCR. Total RNA was isolated from H₂O₂-treated and non-treated HCECi with the RNeasy Micro Kit (Qiagen). Reverse transcription was performed using a commercially available kit (Promega). TaqMan primers and probes for Nrf2 and for the endogenous control B2M were obtained from Applied Biosystems. Real-time PCR reactions were run on an ABI Prism 7500 HT Sequence Detection System (Applied Biosystems). For data analysis, the comparative Ct method was performed.

Corneal Whole Mount Assays. Eight (8-) to 12-wk-old male BALB/c and C57BL/6 (from Taconic Farms, Germantown, N.Y. and our own breeding facility) were used for corneal whole mount assays. All protocols were approved by the Schepens Eye Research Institute Animal Care and Use Committee, and all animals were treated according to the Association for Research in Vision and Ophthalmology Statement for the Use of Animals in Ophthalmic and Vision Research. Whole mount corneas, endothelial cell side up, dissected from mice, were stored in serum free DMEM and subjected to various concentrations of H₂O₂ (0-100 μM) for variable time periods (30 minutes to 12 hours) at 37° C. Control corneal buttons were incubated in DMEM only at 37° C. for 0-12 hours.

Immunocytochemistry. Immunocytochemistry was performed as previously described.17 Normal, FECD, and PBK corneas were incubated in culture medium containing 100 nM MitoTracker Red CMXRos (Molecular Probes) for 30 minutes at 37° C., then fixed with 3.7% formaldehyde. After permeabilization, corneas were incubated with goat anti-8-OHdG (1:200; Millipore) followed by incubation with appropriate secondary antibodies. Nuclei were stained with TO-PRO-3 iodide (Molecular Probes). Corneal endothelial tight junctions were detected by rabbit anti-zonula occludens-1 (anti-ZO-1) antibody (1:300; Invitrogen). Apoptosis was detected using annexin-V-FITC (1:20; Bioscience), propidium iodide (1:200; Invitrogen), and TUNEL assay (In Situ Cell Death Detection Kit, Roche Diagnostics GmbH) according to the manufacturers' instructions. Digital images were obtained using a spectral photometric confocal microscope (Leica DM6000S with LCS 1.3.1 software).

Analysis of CE Cell Morphology. Images of corneal endothelium after staining with anti-ZO-1 were uploaded into Confoscan 4 (NIDEK Technologies) microscope, which performs automatic cell analysis. The software was used to detect the number of cell sides, the area of each cell, and endothelial cell density; polymegethism (variation in cell size) and pleomorphism (variation in cell shape) were then calculated.

Statistical Analysis. Data were analyzed with the statistical analysis software SPSS 16.0. Statistical analyses were performed using a two-tailed unpaired Student's t test, and one-way analysis of variance (ANOVA) with significant differences between groups identified using least—squares difference (LSD) post hoc test. Results were expressed as mean±standard error of the mean (SEM) and considered significant at P<0.05.

### Oxidant-Antioxidant Imbalance in FECD CE

Experiments were carried out to determine whether PRDX synthesis is the only antioxidant family affected in FECD, and whether there is alteration of expression or compensatory overexpression of other antioxidants in FECD CE. Human Oxidative Stress and Antioxidant Defense RT2-PCR Arrays comparing native normal and FECD endothelial cells were employed. The samples were age and sex matched (Table I). A change in mRNA expression of more than 2-fold in FECD relative to normal was set as the cutoff value for considering a gene to be underexpressed or overexpressed. The PCR array included 84 oxidative stress-related genes, of which 61 (73%) had detectable expression in human corneal endothelium. Of the 61 genes detected, 18 transcripts (30%) were more than 2-fold underexpressed, and two (3%) were more than 2-fold overexpressed, as shown in the scatter plot (FIG. 1A). Of the underexpressed genes, statistical significance (P<0.05) was noted in five antioxidant genes—two genes related to apoptosis, two involved in cell signaling, and one involved in oxidative stress response (Table II). The two overexpressed genes were albumin (ALB) and neutrophil cytosolic factor 2 (NCF2).

A diagram depicting the main antioxidant enzyme systems responsible for reactive oxygen species (ROS) metabolism to water is shown in FIG. 1B. The antioxidants found to be dysregulated in FECD are indicated with arrows. Transcriptional downregulation of PRDX genes was confirmed. In addition, downregulation of thioredoxin reductase 1 (TXNRD1), a reductant necessary to replenish PRDX activity, and metallothionein 3 (MT3), a potent ROS scavenger, were noted. There was a decrease in two superoxide dismutase (SOD) isoforms, but only SOD2, a mitochondrial antioxidant, was downregulated at a statistically significant level. Compensatory overexpression of ubiquitous antioxidants such as catalase and glutathione peroxidases, or any other antioxidants involved in ROS scavenging were not detected.

To determine whether there is a common transcriptional regulation of the underexpressed antioxidants in FECD, a computer-based search for ARE (TGCTGA(G/C)TCAGCA) in the promoter region of the genes, up to 5 kb upstream, was carried out. Fifteen (15) out of 18 downregulated genes (above the 2-fold cutoff) in FECD had an ARE sequence in the proximal promoter regions (Table 2).

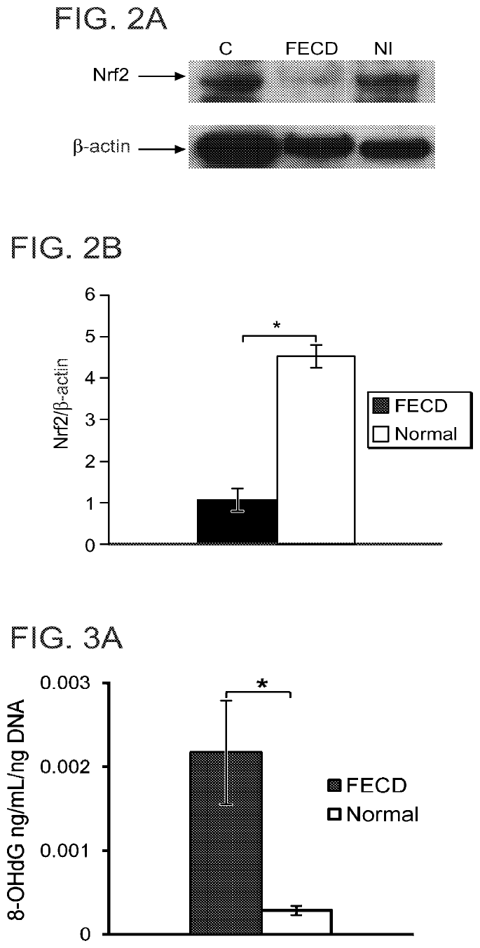

The transcription factor nuclear factor-erythroid 2-related factor 2 (Nrf2) is known to bind the ARE sequence and cause a coordinated upregulation of antioxidant and xenobiotic-metabolizing enzyme genes during oxidative stress. To examine the involvement of the Nrf2 transcription factor in FECD, we compared the levels of Nrf2 between normal and FECD CE. We detected a 4.5-fold decrease (P=0.02) in Nrf2 protein production in FECD CE as compared to normal controls (FIG. 2).

TABLE 2
Genes with More than 2-fold Down- or Upregulation in FECD Corneal
Endothelium Relative to Normal, as Detected by PCR Array

| | | | | |
|---|---|---|---|---|
| | | Fold | | ARE^{†} site |
| Gene description | Symbol | regulation | P value | in promoter |

>2-fold downregulated

| | | | | |
|---|---|---|---|---|
| Antioxidant | | | | |
| Metallothionein 3 | MT3 | −5.65 | 0.02 | x |
| Superoxide dismutase 3, extracellular | SOD3 | −5.37 | 0.10 | x |
| Peroxiredoxin 2 | PRDX2 | −4.30 | 0.04 | x |
| Peroxiredoxin 6 | PRDX6 | | 0.06 | x |
| Peroxiredoxin 5 | PRDX5 | −3.05 | 0.01 | x |
| Superoxide dismutase 2, mitochondrial | SOD2 | −2.65 | 0.00 | x |
| Cytoglobin | CYGB | −2.23 | 0.16 | x |
| Thioredoxin reductase 1 | TXNRD1 | −2.23 | 0.02 | x |
| Peroxiredoxin 1 | PRDX1 | −2.02 | 0.19 | x |
| ROS metabolism | | | | |
| Nitric oxide synthase 2A (inducible, hepatocytes) | NOS2A | −3.91 | 0.22 | |
| Arachidonate 12-lipoxygenase | ALOX12 | −2.31 | 0.40 | |
| Apoptosis | | | | |
| BCL2/adenovirus E1B 19 kDa interacting protein 3 | BNIP3 | −4.61 | 0.00 | x |
| Glyceraldehyde-3-phosphate dehydrogenase | GAPDH | −2.61 | 0.03 | |
| Signaling in response to oxidative stress | | | | |
| Dual specificity phosphatase 1 | DUSP1 | −3.12 | 0.81 | x |
| Oxidative-stress responsive 1 | OXSR1 | −2.18 | 0.83 | x |
| Serine/threonine kinase 25 (STE20 homolog, yeast) | STK25 | −2.16 | 0.11 | x |
| Other oxidative stress responsive genes | | | | |
| Angiopoietin-like 7 | ANGPTL7 | −4.74 | 0.02 | x |
| Selenoprotein P, plasma, 1 | SEPP1 | −2.03 | 0.07 | x |

>2-fold upregulated

| | | | | |
|---|---|---|---|---|
| Antioxidant | | | | |
| Albumin | ALB | 3.34 | 0.15 | |
| ROS metabolism | | | | |
| Neutrophil cytosolic factor 2 | NCF2 | 3.48 | 0.02 | x |

^{†}ARE: antioxidant-responsive element.
x, gene that has the ARE consensus sequence in his promoter sequence.
Note:
Boldfaced genes had statistically significant fold changes in FECD CE as compared to normal.

### Increased Oxidative DNA Damage in FECD CE

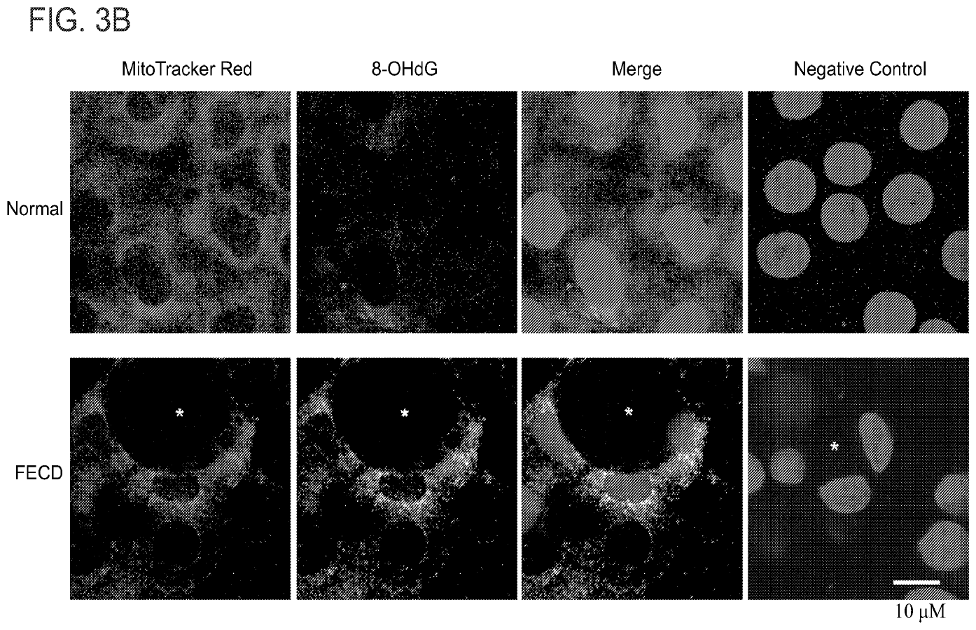

To determine whether alterations in the antioxidant gene profile are accompanied by oxidant-mediated injury to the cell, levels of oxidative DNA damage in FECD endothelium were assayed. Accumulation of oxidized DNA is characteristic of ROS-induced molecular damage during aging and in pathological conditions that are linked to oxidative stress. One of the major DNA base lesions is 8-OHdG. A competitive ELISA was used to compare 8-OHdG levels between FECD and normal corneal endothelium. FIG. 3A shows the average concentration of 8-OHdG normalized to the amount of DNA loaded in FECD patients and normal controls. The FECD group had 2.17E-03±6.01E-04 ng/ml 8-OHdG per ng of DNA, and the normal group 2.90E-04±3.71E-05 ng/ml. Thus, the average level of 8-OHdG in FECD was 7.5-fold higher than in normal samples (P=0.006). These results indicate that oxidative DNA damage is increased in FECD CE compared to age- and sex-matched controls.

### Colocalization of Oxidative DNA Damage and Mitochondria

In order to determine whether oxidative DNA damage was in mitochondria or nuclei, normal and FECD CE was labeled with anti-8-OHdG antibody and MitoTracker, a mitochondrial selective probe that is taken up by mitochondria before cell fixation (FIG. 3B). In normal endothelium, the mitochondria (red) are present throughout the cytoplasm, with greater aggregation around the nuclei (blue). Minimal binding of the anti-8-OHdG antibody (green) is noted. In FECD CE, a characteristic rosette-type clustering of endothelial cells occurs around dark centers representing the guttae. The total number of cells is diminished in FECD due to dystrophic degeneration (bottom row), as compared to normal tissue (top row). In diseased cells, the mitochondria-specific stain is present predominantly around the nuclei. There is significant binding of the anti-8-OHdG antibody primarily around the nuclei, which cluster around the guttae. Colocalization of 8-OHdG with mitochondria is clearly indicated by yellow fluorescence. These findings provide evidence that it is primarily mitochondrial DNA that sustains oxidative damage in FECD-affected endothelium.

To assess whether molecular alterations seen in FECD tissue are induced by oxidative stress, immortalized human corneal endothelial cells (HCECi) were treated with H₂O₂. Following a 2-hour treatment with H₂O₂, there was a statistically significant decrease in Nrf2 transcription (P=0.004) in treated cells compared to non-treated controls. The mean±SEM relative expression of Nrf2 mRNA was (0.66±0.02) and (1.00±0.05), respectively. Concomitantly, the level of 8-OHdG in cells exposed to H₂O₂ was 2-fold higher than in control cells (P=0.001). These in vitro studies indicate that oxidative stress causes a downregulation of Nrf2 along with an increased amount of oxidative DNA damage in vitro, thus mimicking the changes seen in native diseased samples.

### Colocalization of Oxidative DNA Damage and Apoptosis is Specific to FECD

Another major reason for CE cell loss and development of edema in human cornea is pseudophakic bullous keratopathy (PBK), a disease caused by iatrogenic damage to CE during surgery, mainly cataract extraction. To evaluate whether apoptotic cell death and oxidative damage are specific to FECD, level of apoptosis and oxidative damage were compared between normal, FECD and PBK human specimens by labeling with terminal deoxynucleotidyl transferase Biotin-dUTP nick end labeling (TUNEL) and anti-8-OHdG antibodies. Total number of CE cells per mm² was significantly lower in the specimens taken from FECD (933±253, P=0.04) and PBK (718±313, P=0.04) donors, as compared to normal controls (2079±440) (FIGS. 5A,B). Significantly more TUNEL-labeled apoptotic cells were detected in FECD specimens compared to normal (P=0.017) samples, whereas TUNEL binding was not statistically significant between normal and PBK (P=0.54) samples (FIG. 5A, B). In addition, oxidative DNA damage, as indicated by 8-OHdG immunolocalization, was not detected in PBK corneas, while it colocalized with TUNEL-positive apoptotic cells in FECD specimens (FIG. 5A). Densitometric analysis of confocal images detected increased 8-OHdG labeling in FECD as compared to normal (P=0.0293) and PBK (P=0.004) specimens (FIG. 5C).

### Oxidative Stress Induces CE Cell Polymegethism and Pleomorphism

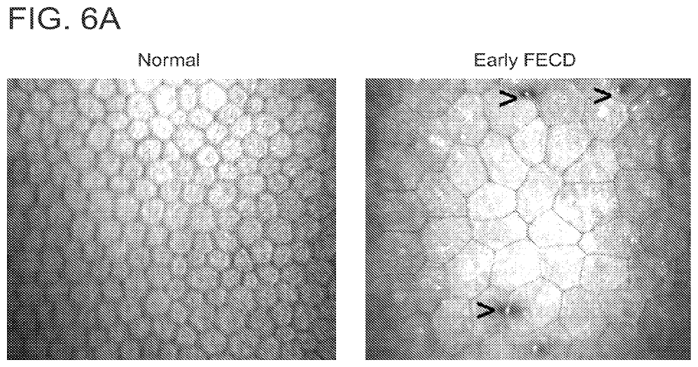

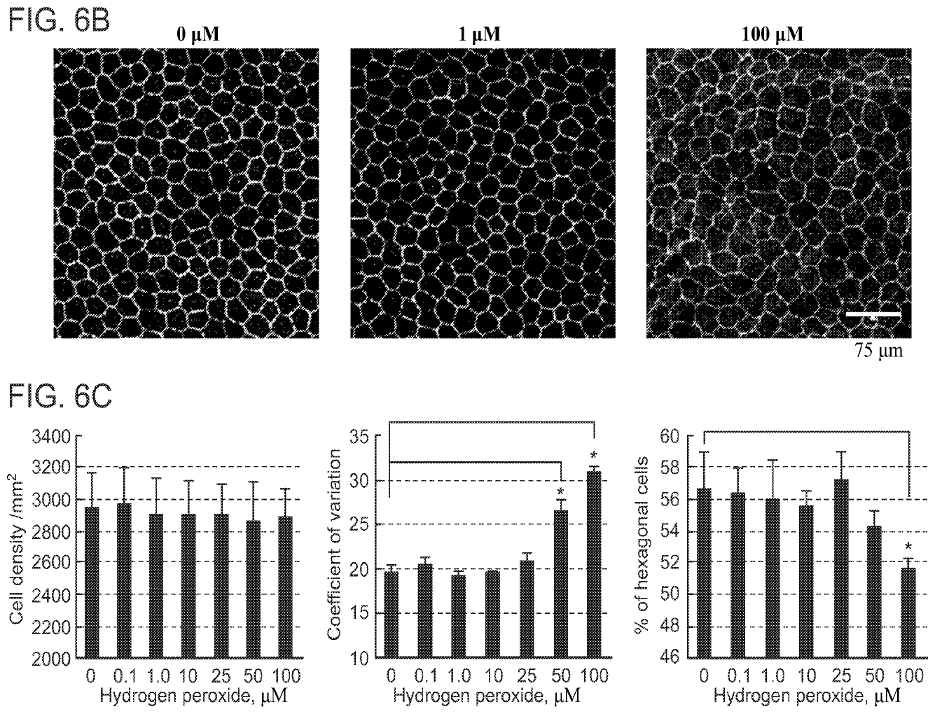

To investigate the role of oxidative stress on CE cell morphological changes, denoted as polymegethism and pleomorphism, which are seen in FECD clinically (FIG. 6A), excised mouse corneas with endothelial side up were treated with H₂O₂ in the corneal whole mount assay. CE tight junctions were labeled with anti-ZO-1 antibody and Confoscan 4 software was used to determine endothelial cell density and morphological changes due to oxidative stress ex vivo (FIG. 6B). Normal mouse CE cell density per mm² was 2943±221, coefficient of variation (measure of polymegethism) 19.6%±0.83, and percent of hexagonal cells (measure of pleomorphism) was 56.6%±2.4 (FIG. 6C). A statistically significant change in polymegethism was induced after 50 μM (P<0.001) and 100 μM (P<0.001) treatments, while the percent of hexagonal cells declined significantly after 100 μM treatments (P=0.007) (FIG. 6C). Therefore, H₂O₂ treatments induced a dose-dependent increase in polymegethism and an increase in pleomorphism (FIG. 6C). There was no statistically significant effect of pro-oxidant treatments on CE cell density as measured by ZO-1 labeling.

### Corneal Endothelial Cell Apoptosis and Loss of Mitochondrial Membrane Potential Due to Oxidative Stress Ex Vivo

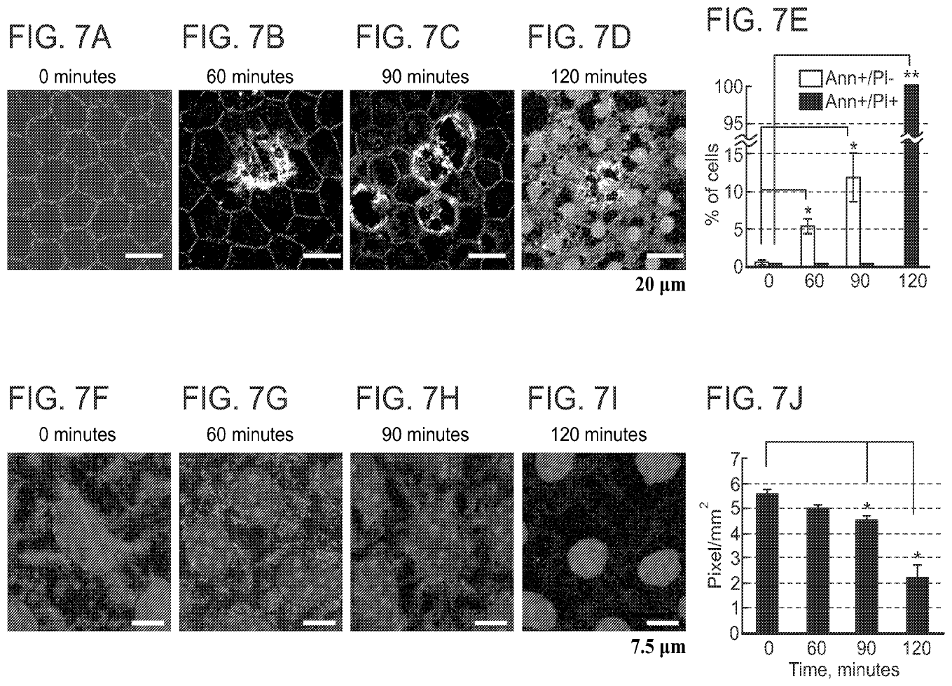

To evaluate the role of oxidative stress on CE cell apoptosis in ex vivo corneal organ culture, where endothelium is attached to its native basement membrane, excised mouse corneas were treated with low-dose (1 μM) H₂O₂. Labeling of exposed phosphatidylserine on the outer phospholipid leaflet of the plasma membrane by annexin-V antibodies enabled visualization of the time-dependent onset of CE cell early apoptosis (FIG. 7B-D). Approximately 5% and 11% of CE cells exhibited early apoptosis as detected by annexin-V-positive and PI-negative staining at 60 minutes (P<0.01) and 90 minutes (P<0.01), respectively (FIG. 7E). Late apoptosis, as detected by annexin-V-positive and PI-positive staining, was identified at 120 minutes (P<0.01). Necrosis, detected by PI staining without annexin-V labeling, constituted a very minimal component of CE death due to low-grade oxidative stress. Corneal buttons incubated for the same time periods in only DMEM did not exhibit a statistically significant increase in apoptotic cell death.

To investigate whether there is an alteration of CE cell mitochondrial viability due to oxidative stress (H₂O₂, 1 μM), CE cells ex vivo were labeled with a MitoTracker CMXRos probe. Mitochondria showed dense staining with MitoTracker in the untreated corneas (FIG. 7A), while a decline in the mitochondrial density was identified after the 1-hour treatment (FIG. 7G). A significant decrease in detectable mitochondrial staining was present after 90 minutes (P<0.01) (FIG. 7H), and no detectable mitochondrial staining was noted after the 2-hour H₂O₂ treatment P<0.01) (FIG. 7I). There was a time-dependent loss of mitochondrial density due to low-grade oxidative stress (FIG. 7J). The timing of initial decline in mitochondrial staining (between 60 and 90 minutes) correlated with early apoptotic changes. Late apoptosis (2 hours) correlated with the absence of mitochondria-specific dye uptake into CE and possibly complete loss of mitochondrial membrane potential.

The presence of oxidative damage in FECD corneal endothelium and concomitant modulation of the antioxidant gene profile indicates that oxidative stress is an important contributor to the corneal endothelial morphological changes, apoptosis, and subsequent degeneration in FECD.

Proteomic analysis demonstrated a deficiency in PRDX antioxidants in FECD-affected corneal endothelium. The PCR array data presented herein, comparing native normal and FECD CE detected transcriptional downregulation of PRDX genes, confirmed the proteomic data. In addition to their antioxidant properties in scavenging H₂O₂, PRDXs are known to regulate H₂O₂ signaling, serve as molecular chaperones and function as anti apoptotic molecules. Therefore, cellular protective mechanisms may be lost due to the downregulation of PRDX genes in FECD.

The PCR array was performed to determine whether antioxidant genes other than PRDX, were affected in FECD. A decrease was detected in other antioxidants, such as SOD2, MT3, and TXNRD1; the latter being involved in restoring reducing equivalents required for PRDX enzymatic activity (FIG. 1B). Several genes involved in apoptosis and signaling in response to oxidative stress were also downregulated, as shown in Table 2. Surprisingly, no compensatory increase in the level of antioxidants, such as catalase or glutathione peroxidases and/or transferases, was observed. In addition, we did not detect a compensatory increase in level of sulfiredoxin, which is known to ‘rescue’ PRDXs by preventing their hyperoxidation under oxidative stress. Such generalized downregulation (and lack of upregulation) of antioxidants in FECD, points to diminished transcriptional activation of the promoter sites common to cellular antioxidant defense, as discussed below. The only statistically significantly overexpressed enzyme was NCF2, which is known to be involved in excessive ROS generation in pathologic conditions. The results indicate that there is a generalized downregulation of the oxidative stress-related genes, tipping the oxidant-antioxidant balance towards a pro-oxidant state in FECD.

In response to oxidative stress, cells function to counteract the oxidant effects and restore redox balance by activating or silencing genes encoding defensive enzymes, transcription factors, stress-induced proteins, and apoptotic pathways. Studies were therefore carried out to investigate whether a common transcriptional mechanism could be responsible for the underexpression (or lack of upregulation, as in the case of glutathione peroxidases, sulfiredoxin, and catalase) of the antioxidant defense in FECD. The downregulated antioxidants in FECD were found to contain ARE in their proximal promoter regions. Activation of antioxidants such as PRDXs, TXNRDs, SODs, and MT3 is dependent on Nrf2 transcription factor via binding of the ARE. Nrf2 belongs to the Cap'n'Collar family of transcription factors that bind ARE sequence and cause a transcriptional upregulation of antioxidants in response to oxidative stress. A decrease in Nrf2 protein production in FECD samples was detected as compared to normal controls. Such a decrease in Nrf2 protein level, along with evidence of Nrf2 antioxidant target decline in FECD, provides evidence that there is dysregulation of Nrf2-regulated constitutive expression of multiple antioxidants in FECD corneal endothelium.

Nrf2-ARE-driven gene activation protects neuronal cells from H₂O₂-induced apoptosis. The cellular mechanisms for counteracting oxidative stress involve upregulation, stabilization, and nuclear translocation of Nrf2. Given the extensive oxidative DNA damage in FECD, it was possible that Nrf2 would be upregulated in FECD in response to oxidative injury. However, a decline in Nrf2 protein in FECD might suggest an aberrant Nrf2 response in the diseased cells. Similarly, studies have not detected Nrf2 activation in Alzheimer's disease brains despite evidence of oxidative stress in the neuronal cells. Nrf2 deficiency has been demonstrated to enhance susceptibility of fibroblasts to photooxidative damage and of lung tissue to cigarette smoke. The data confirmed, in an in vitro model using corneal endothelial cells, that H₂O₂ can cause concomitant downregulation of Nrf2 and an increase in DNA oxidative damage. Therefore, the dysregulated Nrf2-controlled pathway is of critical relevance to understanding the cellular and molecular mechanisms that cause endothelial cell oxidative damage and, potentially, apoptosis in FECD. Targeting Nrf2, which upregulates a wide range of antioxidants and confers cytoprotection in other oxidative stress-related disorders, is an attractive pharmacologic strategy for treating corneal endothelium.

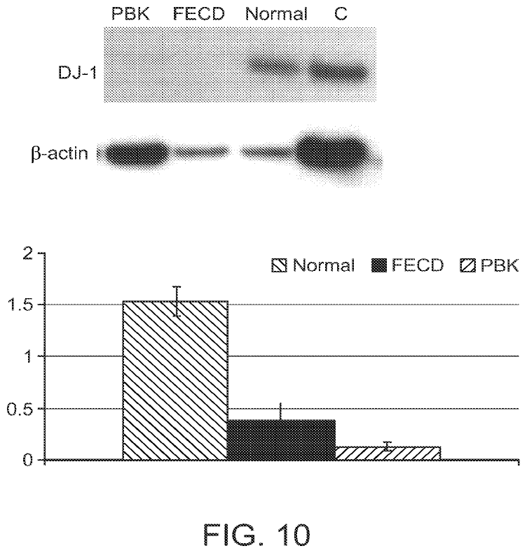

Oxidative stress in a cell leads to DNA damage. Increased levels of 8-OHdG, a marker of oxidative damage to DNA, we found in FECD CE as compared to age-matched controls. 8-OHdG is a DNA adduct that accumulates over the lifespan of an individual, primarily in mitotically fixed and metabolically active tissues, and cell types such as brain and corneal endothelium. The finding of elevated levels of oxidized guanosine base in FECD is particularly relevant and important, and, for the first time, places FECD in the category of oxidative stress-related disorders. Furthermore, the study detected that mitochondrial DNA (mtDNA) is the primary target of oxidative damage in FECD. The mitochondrial respiratory chain is a major internal source of ROS production. Thus, mitochondria accumulate oxidative damage more rapidly than the rest of the cell. mtDNA is particularly susceptible to oxidative damage due to several factors; it is located close to the ROS-generating respiratory chain, it is not covered by histones, and it lacks a strong repair system compared to that of nuclear DNA. The fact that downregulation of key mitochondrial antioxidants, such as SOD2 and PRDX5 in FECD, was detected points to potentially higher susceptibility of mtDNA to oxidative stress in the disease state. mtDNA damage is known to cause dysfunctional mitochondrial protein synthesis, loss of integrity of inner mitochondrial membrane potential, and apoptotic cell death. There are decreased numbers of mitochondria in FECD endothelium, and that activity of cytochrome oxidase, the major respiratory chain enzyme, is decreased in the central area of FECD corneal buttons. The studies show that oxidative stress-induced mtDNA damage leads to a decrease in mtDNA transcription of the major electron transport chain proteins in FECD. Moreover, the data indicate that that there is oxidant-antioxidant imbalance in Fuchs endothelial corneal dystrophy (FECD) and pseudophakic bullous keratopathy (PBK). For example, DJ-1 protein, a major antioxidant and a positive regulator of Nrf2, was found to be downregulated in FECD (p<0.001) and PBK (p<0.001) (FIG. 10).

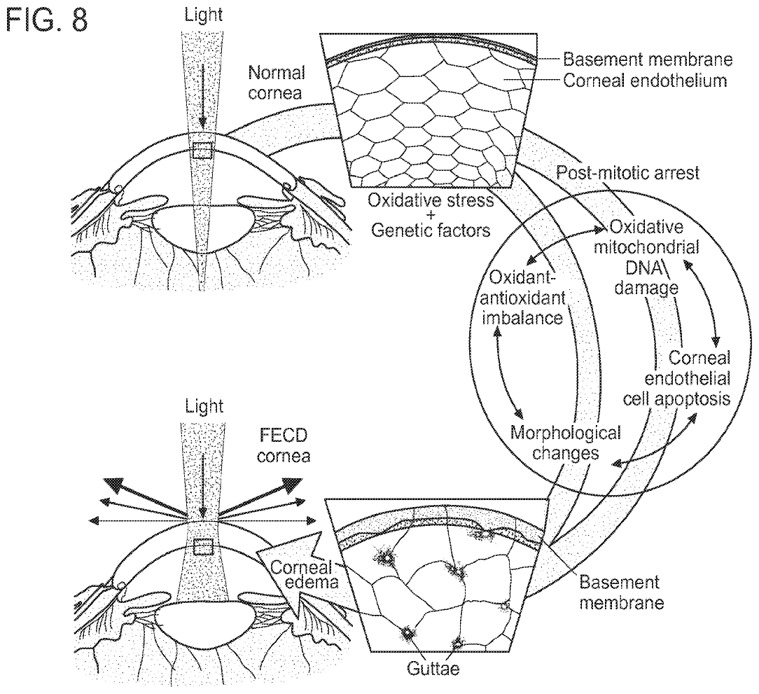

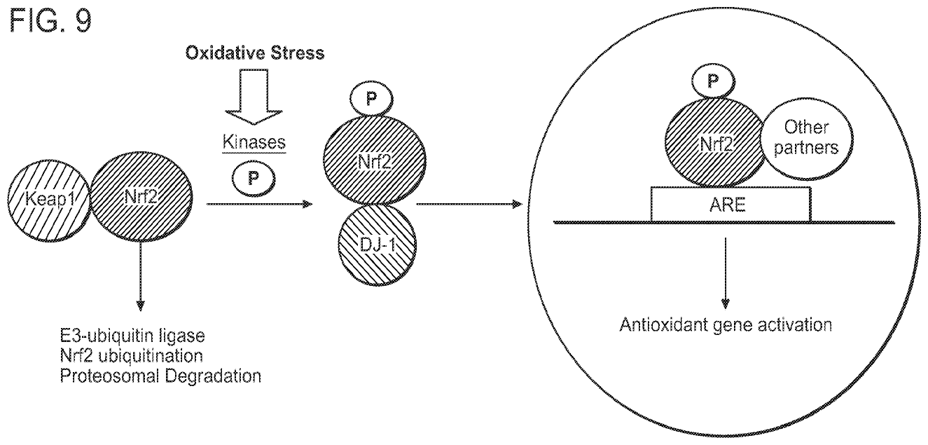

The key characteristic features of FECD are apoptotic cell death and aberrant extracellular matrix deposition that manifests in disruption of the hexagonal CE cell mosaic. The studies on native FECD specimens correlate apoptotic cell death and oxidative damage. These findings are specific to the dystrophic degeneration and do not occur in PBK, a condition that also manifests in CE cell loss and corneal edema. Based on the studies on ex vivo corneas, oxidative stress induces morphological alterations in endothelial cell size and shape that mimic the changes seen in FECD. In addition, oxidative stress-induced CE cell loss of mitochondrial membrane potential correlated with the onset of early and late apoptosis in the ex vivo setting, thus pointing to a potential mechanism for endothelial cell loss in FECD. A schematic representation of the pathogenesis of FECD is shown in FIG. 8. The protein and gene profiling studies, along with the identification of the intracellular oxidative damage in the endothelium affected by the dystrophy, addresses the interaction of genetic factors (which are mostly unknown in FECD at this time) and environmental factors, thus enabling characterization of the pathogenic mechanisms that eventually lead to loss of CE cells and corneal edema.

### Example 2: Nrf2 Agonists Such as Sulforaphane and D3T Diminish Corneal Endothelial Cell Apoptosis and Prevent Cell Loss Under Normal and Pro-Oxidant Conditions

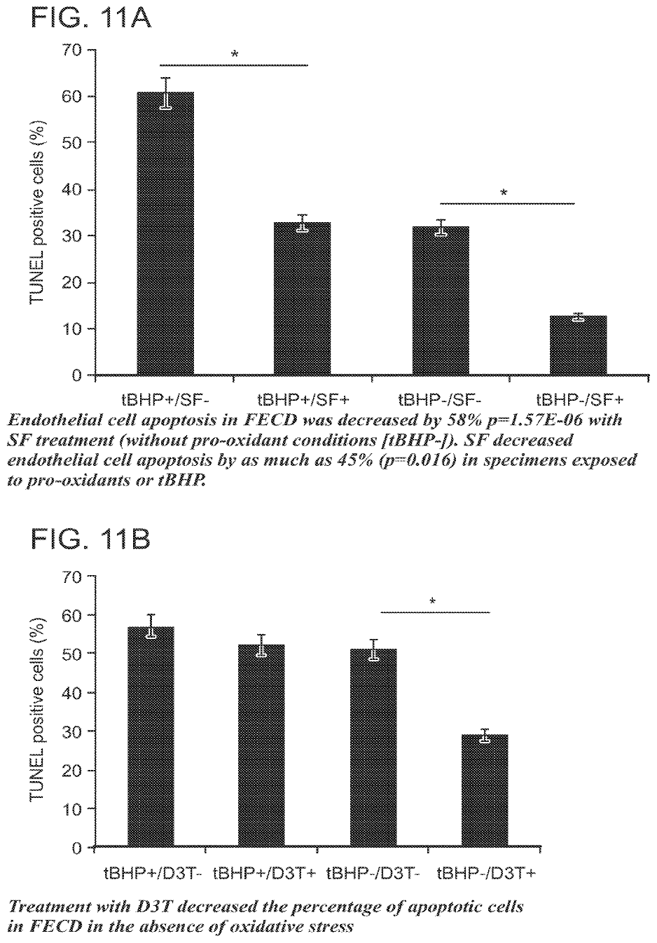

An Nrf2-controlled pathway is affected during the endothelial cell loss from surgery and from dystrophic degeneration. Levels of antioxidants that are regulated by Nrf2 transcription factor were found to be decreased in diseased corneal endothelium. Nrf2 is a transcription factor that causes a coordinated upregulation of multiple antioxidants, such as glutathione transferases, glutathione peroxidases, peroxiredoxins, thioredoxins, NADH(P)H hydrogenases, heme-oxygenases, glutamate-cysteine ligases. Sulforaphane (SF) and D3T upregulate Nrf2 on a protein level and enhance cellular antioxidant defense. Thus, studies were carried out to investigate the effect of SF on endothelial cell apoptosis with and without pro-oxidants. Post-keratoplasty FECD specimens, containing corneal endothelium attached to the Descemet membrane, were exposed to four experimental conditions: +/−tBHP with is a long acting pro-oxidant and +/−SF. The rate of endothelial cell apoptosis (as detected by TUNEL) in FECD endothelium was 31% and it decreased to 13% (p=1.5E-06) with SF treatment. Under pro-oxidant conditions, SF decreased endothelial cell apoptosis from 60% to 33% (p=0.016) (FIG. 11A). Similarly, D3T diminished endothelial cell apoptosis by decreasing the number of apoptotic cells from 52% to 30% (p<0.05) (FIG. 11B).

A human tissue-based model was used to study the mechanism of the disease process that most closely resembles the in vivo situation. SF was found to have a potent effect on rescuing diseased endothelial cells from apoptosis at baseline and when exposed to an oxidizing environment. Therefore, Nrf2 agonists or activators arrest the disease process in early as well as late FECD and prevent loss of endothelium as seen during intraocular surgery.

A commonly used intraocular irrigating solution during ocular surgery is BSS PLUS® from Alcon. This solution contains electrolytes and two antioxidants, mainly, glutathione and ascorbate. It has been well established that oxidative stress is involved in endothelial cell loss during intraocular surgery and in endothelial cell loss in FECD. The data described herein provide evidence that upregulation of multiple antioxidants and cytoprotective enzymes is an attractive pharmacologic strategy since a single antioxidant molecule such as a vitamin is not efficacious or potent enough to ameliorate a robust oxidant-antioxidant imbalance seen in FECD and PBK. Thus, the addition of Nrf2 agonists or activators to such irrigating solutions serves to reduce oxidative stress an minimize endothelial cell loss during surgical or other manipulation of ocular tissue.

The data demonstrate that there is downregulation of ARE-driven antioxidant and oxidative stress-related gene expression, a decline in the levels of the major transcription factor known to regulate ARE-dependent antioxidants, and an increase in oxidative mtDNA damage in FECD. The suboptimal Nrf2-regulated antioxidant defense most likely contributes to the oxidant-antioxidant imbalance seen in FECD. Based on the findings of this study, oxidative stress directly associates with FECD pathogenesis and is a novel and attractive target for therapy development for this common ocular condition. The methods and composition described herein prevent corneal endothelial cell loss in early as well as late stages of the disease by reversing oxidant-antioxidant imbalance thereby delaying or bypassing completely the need for corneal transplantation.

1-22. (canceled)

23. A method for treating a corneal endothelium disorder in a subject, comprising administering to the subject a therapeutically effective amount of a composition comprising a vector expressing Nrf2, thereby treating the corneal endothelium disorder.

24. The method of claim 23, wherein the vector is packaged in a virus-based expression system selected from a parvovirus expression system; a lentivirus (LV) expression system; a retrovirus expression system; an adenovirus expression system; a herpesvirus expression system; an adeno-associated virus (AAV) expression system; a viral-mediated RNAi (siRNA) expression system; AdV-mediated expression of shRNA; LV-based delivery of shRNA; and viral-based reversible and irreversible regulated delivery of shRNA retroviral vector.

25. The method of claim 23, wherein the disorder is selected from Fuchs endothelial corneal Dystrophy (FECD), posterior polymorphous dystrophy, congenital hereditary endothelial dystrophy (CHED), iridocorneal endothelial (ICE) syndrome, and pseudophakic bullous keratopathy (PBK).

26. The method of claim 23, wherein the composition is administered systemically.

27. The method of claim 23, wherein the composition is administered locally to the vitreous, the subretinal space and/or the sub-tenar capsule.

28. The method of claim 23, wherein the composition comprises the vector and a pharmaceutically acceptable fluid.

29. The method of claim 23, wherein the composition is administered before, during, or after an intra-ocular surgery, phacoemulcification or a vitreoretinal procedure.

30. The method of claim 23, wherein the composition is administered by microinjection, infusion, scrape loading, or electroporation.

31. The method of claim 23, wherein said subject is a human subject.

32. A nanoparticle comprising an effective amount of a composition comprising a vector expressing Nrf2.
